(19) United States
(12) Patent Application Publication
(10) Pub. No.: US 2021/0293797 A1
(43) Pub. Date: Sep. 23, 2021

(54) METHOD FOR DETECTING THE PRESENCE OF ONE OR MORE BACTERIAL TOXINS IN A BIOLOGICAL FLUID USING LIPOSOMES

(71) Applicant: The University of Liverpool, Liverpool (GB)

(72) Inventors: Aras Kadioglu, Liverpool (GB); Daniel Neill, Liverpool (GB); Stavros Panagiotou, Liverpool (GB)

(21) Appl. No.: 16/612,198

(22) PCT Filed: May 10, 2018
(86) PCT No.: PCT/GB18/51261

§ 371 Date: Nov. 8, 2019

(30) Foreign Application Priority Data
May 11, 2017 (GB) 1707559.9

(51) Int. Cl. G01N 33/543; G01N 33/569; G01N 33/58

(52) U.S. Cl. CPC G01N 33/5432; G01N 33/54306; G01N 2800/709; G01N 33/582; G01N 33/56944

(57) ABSTRACT

The present invention relates to a method for detecting the presence of one or more bacterial toxins, capable of binding to cell membranes, in biological fluid wherein the method comprises: (i) incubating the biological fluid with a plurality of liposomes, wherein the liposomes comprise a lipid capable of binding to said one or more toxins, to provide one or more liposome-toxin conjugate; (ii) incubating said conjugates with at least one type of antibody bound to a label to provide one or more conjugate-antibody complex; wherein each type of antibody in the mixture is specific for one of the bacterial toxins whose presence is to be detected; and (iii) analysing said complexes in order to detect the presence of one or more bacterial toxins capable of binding to cell membranes. Further aspects of the invention relate to a conjugate-antibody complex useful in the methods of the invention and a kit useful for performing the method of the invention.

### INTRODUCTION

The present invention relates to a method for detecting the presence of one or more bacterial toxins in a biological fluid using liposomes. In particular, the present invention provides a method for diagnosing diseases triggered and exacerbated by bacterial toxins, such as sepsis.

### BACKGROUND OF THE INVENTION

Sepsis costs the UK National Health Service (NHS) £2.3 billion per year and causes tens of thousands of deaths (Daniels R., The incidence, mortality and economic burden of sepsis (2009) In: NHS Evidence emergency and urgent care. http://library.nhs.uk/Emergency). It has been estimated in European studies that a typical episode of severe sepsis costs a healthcare organisation approximately €25,000 (Vincent J L, Sakr Y, Sprung C L, et al. Sepsis in European intensive care units: results of the SOAP study, Critical Care Medicine 2006; 34: 344-353). The greatest burden of human disease is caused by bacterial pathogens that secrete cytolytic, membrane-damaging toxins that play a critical role in the establishment and progression of infections, such as infections seen during bacterial sepsis. These include: [1] Streptococcus pneumoniae (Sp) and its toxin pneumolysin, [2] Staphylococcus aureus (Sa) and alpha-toxin, [3] Streptococcus pyogenes (Spy) and streptolysin O, [4] Pseudomonas aeruginosa (Pa) and exotoxin A, [5] Escherichia coli (Ec) and alpha-haemolysin and [6] Klebsiella pneumonia (Kp) and haemolysin.

Clinical decisions regarding choice of empiric antibiotic therapy require knowledge of the causative agent of infection and the likely susceptibility of the pathogen to antibiotics. Identifying a causative agent in cases of bacterial sepsis is difficult, relying on time-consuming molecular diagnostics such as Maldi-Tof and multiplex PCR or low sensitivity culture methods.

Correct recognition of the infecting pathogen and rapid treatment is key to survival in patients with sepsis. In the case of septic shock, every hour delay in administration of effective antibiotics is associated with increased mortality (Levy M M, Crit Care Med, 2010). Furthermore, the 2012 International Guidelines for Management of Severe Sepsis and Septic Shock highlighted the importance of positive diagnosis for de-escalation of antimicrobial therapy.

In cases where no positive diagnosis is possible, treatment failure is often due to resistance to initial therapy, something that could be avoided if the causative agent were correctly identified.

Positive pathogen diagnosis would enable duration and dosage of antibiotic treatment to be reduced, helping circumvent problems associated with antimicrobial resistance (AMR).

There is a need in the art for a rapid, more accurate (e.g. reduced likelihood false positives) and sensitive assay for use in cases of suspected bacterial infection to enable the efficient and effective treatment of a range of conditions mediated by bacterial infection, for instance, sepsis.

The present invention is useful in addressing the above unmet need by providing a quick method for detecting/characterising infection. The present invention may also provide a quantitative assessment of infection enabling an indication of patient prognosis as higher levels of toxin mean greater burden of infection which may have a negative correlation with survival.

### SUMMARY OF THE INVENTION

In a first aspect, the present invention relates to a method for detecting the presence of one or more bacterial toxin, capable of binding to cell membranes, in biological fluid wherein the method comprises:

(i) incubating the biological fluid with a plurality of liposomes, wherein the liposomes comprise a lipid capable of binding to said one or more toxins, to provide one or more liposome-toxin conjugate(s);
(ii) incubating said conjugate(s) with at least one type of antibody bound to a label to provide one or more conjugate-antibody complex(es);

wherein each type of antibody in the mixture is specific for one of the bacterial toxins whose presence is to be detected; and

(iii) analysing said complexes in order to detect the presence of one or more bacterial toxins capable of binding to cell membranes.

In a second aspect, the present invention relates to a method for the diagnosis of sepsis wherein the method comprises:

(i) incubating a biological sample from a patient suspected of suffering from sepsis with a plurality of liposomes, wherein the liposomes comprise a lipid capable of binding to one or more bacterial toxin produced by bacteria implicated in the development of sepsis, to provide one or more liposome-toxin conjugate(s);
(ii) incubating said conjugates with at least one type of antibody bound to a label to provide one or more conjugate-antibody complex(es);

wherein each type of antibody in the mixture is specific for a bacterial toxin produced by bacteria implicated in the development of sepsis; and

(iii) analysing said complex(es) in order to detect the presence of bacterial toxins produced by bacteria implicated in the development of sepsis.

In a third aspect, the present invention relates to a method for determining the prognosis of a patient suspected of suffering from sepsis wherein the method comprises:

(i) incubating a biological sample from a patient suspected of suffering from sepsis with a plurality of liposomes, wherein the liposomes comprise a lipid capable of binding one or more bacterial toxin produced by bacteria implicated in the development of sepsis, to provide one or more liposome-toxin conjugate(s);
(ii) incubating said conjugate(s) with at least one type of antibody bound to a label to provide one or more conjugate-antibody complex(es);

wherein each type of antibody in the mixture is specific for a bacterial toxin produced by bacteria implicated in the development of sepsis; and

(iii) analysing said complex(es) in order to detect quantitatively the presence of bacterial toxins produced by bacteria implicated in the development of sepsis.

In a fourth aspect, the present invention relates to a complex comprising (i) a conjugate comprising a liposome and a bacterial toxin; and (ii) an antibody bound to a label.

In a fifth aspect, the present invention relates to a kit for detecting the presence of bacterial toxins comprising:

(i) a container comprising liposomes and optionally a buffer, wherein the liposomes comprise a lipid capable of binding to one or more bacterial toxins; and
(ii) a container comprising a reagent, wherein the reagent comprises at least one type of antibody bound to a label; and
(iii) optionally further containers comprising a reagent, wherein the reagent comprises at least one type of antibody bound to a label;
wherein each type of antibody in the kit is specific for a different bacterial toxin to be detected.

### BRIEF DESCRIPTION OF THE DRAWINGS

FIG. 1—Overview of Toxin Detection Test with analysis by flow cytometry. Although only one liposome is depicted, a plurality are added and the ratio of toxin to liposomes determines the fluorescence intensity.

FIG. 2—Overview of the preparation of a standard curve for quantification of bacterial toxins. Known concentrations of toxin are added to liposomes. The resulting mean fluorescent intensity and number of fluorescence-positive liposomes on the flow cytometer can then be compared to that of patient samples and, from this, concentrations of toxin in the samples can be determined.

FIG. 3—Standard curve generated with pneumolysin-spiked plasma. Healthy plasma spiked with known concentrations of pneumolysin was incubated with liposomes for 30 minutes. The isolated liposome-toxin conjugates were then incubated with allophycocyanin (APC)-conjugated anti-pneumolysin antibody for 30 minutes. The resultant conjugate-antibody complexes were detected by APC-fluorescence in a BD FACScalibur flow cytometer running CellQuest Pro acquisition software. The standard curve allows determination of pneumolyin concentration in patient samples by determining the percentage of APC+liposomes and reading of the corresponding point on the standard curve.

FIG. 4—Flow cytometry based detection of pneumolysin. Plasma from a healthy individual was incubated with fluorescein isothiocyante (FTIC)-labelled liposomes (1 μm diameter) for 30 minutes. Samples (C) and (D) had been pre-spiked with 200 ng/ml purified pneumolysin. Liposomes from samples (B) and (D) were further incubated with allophycocyanin (APC)-conjugated anti-pneumolysin antibody for 30 minutes. Liposome bound pneumolysin was detected by APC-fluorescence on a BD FACScalibur flow cytometer. (A) Lipsome detection in heathy plasma. (B) APC-conjugated anti-pneumolysin antibody does not bind liposomes. (C) Pneumolysin does not degrade liposomes. (D) Liposome-bound pneumolysin can be detected with APC-conjugated antibodies. Numbers in the corner of each quadrant represent the percentage of total cells in each sector.

FIG. 5—Determination of pneumolysin concentration in patient plasma samples. Plasma from patients with a diagnosis of sepsis was incubated with liposomes and anti-pneumolysin antibody. Percentage APC+liposomes was determined (A-D), adjusted relative to blank (liposome alone) sample and then a pneumolysin concentration was determined (E) by comparison with a standard curve generated with pneumolysin-spiked plasma. This is compared with a pneumolysin concentration determined by enzyme-linked immunosorbent assay (ELISA) (E).

FIG. 6—Determination of pneumolysin concentration in mouse serum by flow cytometry following inbucations of FITC-lipsomes and the addition of APC-conjugated anti-pneumolysin antibody.

FIG. 7—Blood pneumolysin concentrations and infection outcome in mouse study.

FIG. 8—Assessment of blood streptolysin levels in serum from infected mice after incubation with FITC-lipsomes and the addition of APC-conjugated anti-streptolysin antibody.

### DETAILED DESCRIPTION OF THE INVENTION

### Bacterial Toxin

In one aspect defined above, the present invention relates to a method for detecting the presence of one or more bacterial toxins wherein the method comprises:

(i) incubating the biological fluid with a plurality of liposomes, wherein the liposomes comprise a lipid capable of binding to said one or more toxins, to provide one or more liposome-toxin conjugate(s);
(ii) incubating said conjugate(s) with at least one type of antibody bound to a label to provide one or more conjugate-antibody complex(es);

suitably each type of antibody in the mixture is specific for one of the bacterial toxins whose presence is to be detected; and

(iii) analysing said complexes in order to detect the presence of one or more bacterial toxins capable of binding to cell membranes.

Many bacterial pathogens secrete toxins that kill or damage host cells, for instance, by forming pores (pore-forming toxins) in the host cell membrane or by degrading plasmalemmal lipids (plasmalemmal lipase toxins).

In one embodiment, the one or more bacterial toxins are selected from pore-forming bacterial toxins and plasmalemmal lipase toxins. Suitably, the one or more bacterial toxins are pore-forming toxins.

In one embodiment, the one or more bacterial toxins are bacterial toxins capable of binding to eukaryotic cell membranes. Suitably, the one or more bacterial toxins are bacterial toxins capable of binding to mammalian cell membranes. Suitably, the one or more bacterial toxins are bacterial toxins capable of binding to human cell membranes.

In one embodiment, the one of more bacterial toxins are selected from pore-forming toxins capable of binding to human cell membranes. In another embodiment, the one or more bacterial toxins are selected from plasmalemmal lipase toxins capable of binding to human cell membranes.

In one embodiment, the one or more bacterial toxins are selected from bacterial toxins involved in the aetiology of a disease selected from one or more of sepsis, pneumonia, meningitis and urinary tract infections. Suitably, the one or more bacterial toxins are selected from bacterial toxins involved in the aetiology of a disease selected from sepsis and pneumonia. Suitably, the one or more bacterial toxins are selected from bacterial toxins involved in the aetiology of sepsis.

In a number of aspects above, the one or more bacterial toxin(s) is one or more bacterial toxin(s) implicated in the development of sepsis. Accordingly, at least the following embodiments are relevant.

In one embodiment, the one or more bacterial toxins are selected from bacterial toxins derived from one or more of Streptococcus pneumoniae, Staphylococcus aureus, Escherichia coli, Klebsiella pneumoniae, Pseudomonas aeruginosa, Haemophilus influenzae and Streptococcus pyogenes.

In one embodiment, the one or more bacterial toxins are selected from bacterial toxins derived from one or more of Streptococcus pneumoniae, Staphylococcus aureus, Escherichia coli, Klebsiella pneumoniae, Pseudomonas aeruginosa and Streptococcus pyogenes. Suitably, the one or more bacterial toxins are selected from bacterial toxins derived from one or more of Streptococcus pneumoniae and Staphylococcus aureus. Suitably, the one or more bacterial toxins are selected from bacterial toxins derived from Streptococcus pneumoniae.

In one embodiment, the one or more bacterial toxins are selected from one or more of pneumolysin, alpha haemolysin, haemolysin, exotoxin A and streptolysin O. Suitably, the one or more bacterial toxins are selected from one or more of pneumolysin and alpha haemolysin. Suitably, the one or more bacterial toxins is pneumolysin.

In one embodiment, the one or more bacterial toxins are selected from one or more of pneumolysin (from Streptococcus pneumoniae), alpha haemolysin (from Staphylococcus aureus and/or Escherichia coli), haemolysin (from Klebsiella pneumoniae), exotoxin A (from Pseudomonas aeruginosa) and streptolysin O (from Streptococcus pyogenes). Suitably, the one or more bacterial toxins are selected from one or more of pneumolysin (from Streptococcus pneumoniae) and alpha haemolysin (from Staphylococcus aureus and/or Escherichia coli). Suitably, the one or more bacterial toxins is pneumolysin (from Streptococcus pneumoniae).

In one embodiment, the one or more bacterial toxins are selected from 1, 2, 3, 4, 5, 6, 7, or 8 of pneumolysin, lysteriolysin, tetanolysin, alpha haemolysin, haemolysin, exotoxin A and streptolysin, streptolysin O.

In one embodiment, the one or more bacterial toxins are a combination of pneumolysin, lysteriolysin, tetanolysin, alpha haemolysin, haemolysin, exotoxin A and streptolysin, streptolysin O In one embodiment, the one or more bacterial toxins are a combination of pneumolysin, lysteriolysin, tetanolysin, alpha haemolysin, exotoxin A and streptolysin.

In one embodiment, the one or more bacterial toxins are selected from 1, 2, 3, 4, or 5 of pneumolysin, alpha haemolysin, haemolysin, exotoxin A and streptolysin O

In one embodiment, the one or more bacterial toxins are a combination of pneumolysin, alpha haemolysin, haemolysin, exotoxin A and streptolysin O.

In one embodiment, the one or more bacterial toxins are selected from 1, 2, 3, 4 or 5 of pneumolysin (from Streptococcus pneumoniae), alpha haemolysin (from Staphylococcus aureus and/or Escherichia coli), haemolysin (from Klebsiella pneumoniae), exotoxin A (from Pseudomonas aeruginosa) and streptolysin O (from Streptococcus pyogenes).

In one embodiment, the one or more bacterial toxins are a combination of pneumolysin (from Streptococcus pneumoniae), alpha haemolysin (from Staphylococcus aureus and/or Escherichia coli), haemolysin (from Klebsiella pneumoniae), exotoxin A (from Pseudomonas aeruginosa) and streptolysin O (from Streptococcus pyogenes).

### Biological Fluid

In each of the above aspects, the methods of the present invention are performed on biological fluid.

In one embodiment, the biological fluid is a mammalian biological fluid. Suitably, the biological fluid is a human biological fluid.

In one embodiment, the biological fluid is an ex vivo sample of biological fluid. Suitably, the biological fluid is an ex vivo sample of mammalian biological fluid. Suitably, the biological fluid is an ex vivo sample of human biological fluid.

In one embodiment, the biological fluid is selected from whole blood, blood plasma, blood serum, CSF or urine. Suitably, the biological fluid is selected from whole blood, blood plasma, blood serum or urine. Suitably, the biological fluid is selected from whole blood, blood plasma and blood serum. Suitably, the biological fluid is selected from blood plasma, blood serum or urine. Suitably, the biological fluid is selected from blood plasma.

In one embodiment, the biological fluid is selected from human whole blood, blood plasma, blood serum, CSF or urine. Suitably, the biological fluid is selected from human whole blood, blood plasma, blood serum or urine. Suitably, the biological fluid is selected from human whole blood, blood plasma and blood serum. Suitably, the biological fluid is selected from human blood plasma, blood serum or urine. Suitably, the biological fluid is selected from human blood plasma.

### Liposomes

The methods of the present invention employ liposomes in order to bind or sequester one or more bacterial toxins.

A liposome, as would be known to a person skilled in the art, is a vesicle comprising at least one lipid bilayer. The lipid bilayer may be formed by a variety of liposome-forming lipids. Examples of liposome-forming lipids include without limitation glycerophospholipids and sphingomyelins.

Examples of glycerophospholipids include, without limitation, phosphatidylglycerols (PG) including dimyristoyl phosphatidylglycerol (DMPG); phosphatidylcholine (PC), including egg yolk PC, soy PC, dimyristoyl phosphatidylcholine (DMPC), l-palmitoyl-2-oleoylphosphatidyl choline (POPC), hydrogenated soy phosphatidylcholine (HSPC), distearoylphosphatidylcholine (DSPC); phosphatidic acid (PA), phosphatidylinositol (PI), phosphatidylserine (PS), phosphatiydyl ethanolamine (PE).

Sphingomyelins consist of a ceramide unit with a phosphorylcholine moiety attached to position 1. The phosphocholine moiety in sphingomyelin contributes the polar head group of the sphingomyelin.

In one embodiment, the liposomes comprise other lipids capable of altering the properties of the liposome and/or binding to said one or more toxins, to provide one or more liposome-toxin conjugates. Suitably, said lipids include cholesterol.

In one embodiment, the liposomes comprise cholesterol. Suitably, the liposomes comprise at least about 20 mol. % of cholesterol. Suitably, the liposomes comprise at least about 25 mol. % of cholesterol. Suitably, the liposomes comprise at least about 30 mol. % of cholesterol. Suitably, the liposomes comprise at least about 50 mol. % of cholesterol.

In one embodiment, the liposomes essentially consist of/consist of cholesterol.

In another embodiment, the liposomes comprises 20 mol. % to about 80 mol. % of cholesterol. Suitably, the liposomes comprise about 25 mol. % to about 80 mol. % of cholesterol. Suitably, the liposomes comprise about 30 mol. % to about 80 mol. % of cholesterol. Suitably, the liposomes comprise about 50 mol. % to about 80 mol. % of cholesterol.

Suitably, the liposomes comprises 20 mol. % to about 70 mol. % of cholesterol. Suitably, the liposomes comprise about 25 mol. % to about 70 mol. % of cholesterol. Suitably, the liposomes comprise about 30 mol. % to about 70 mol. % of cholesterol. Suitably, the liposomes comprise about 50 mol. % to about 70 mol. % of cholesterol.

Suitably, the liposomes comprise about 20 mol. % to about 66 mol. % of cholesterol. Suitably, the liposomes comprise about 25 mol. % to about 66 mol. % of cholesterol. Suitably, the liposomes comprise about 30 mol. % to about 66 mol. % of cholesterol. Suitably, the liposomes comprise about 50 mol. % to about 66 mol. % of cholesterol.

In one embodiment, the liposomes comprise cholesterol and sphingomyelin.

In one embodiment, the liposomes essentially consist of cholesterol and sphingomyelin.

In one embodiment, the liposomes consist of cholesterol and sphingomyelin.

In one embodiment, the liposomes comprise between about 1:2 to about 2:1 cholesterol:sphingomyelin (mol %). Suitably, the liposomes comprise between about 1:1 to about 2:1 cholesterol:sphingomyelin (mol %).

In one embodiment, the liposomes essentially consist of between about 1:2 to about 2:1 cholesterol:sphingomyelin (mol %). Suitably, the liposomes essentially consist of between about 1:1 to about 2:1 cholesterol:sphingomyelin (mol %).

In one embodiment, the liposomes consist of between about 1:2 to about 2:1 cholesterol:sphingomyelin (mol %). Suitably, the liposomes consist of between about 1:1 to about 2:1 cholesterol:sphingomyelin (mol %).

In one embodiment, the liposomes comprise ligands capable of binding to one or more bacterial toxin(s). Suitably, the ligands may be peptides or proteins. In one embodiment, the ligand is a lipid receptor, for instance, a sphingosine-1-phosphate receptor.

In one embodiment, the liposomes are multilamellar or unilamellar. Suitably, the liposomes are multilamellar.

In one embodiment, the liposomes are at least about 0.4 μm in diameter. Suitably, the liposomes have a diameter of about 0.4 μm to about 2 μm. Suitably, the liposomes have a diameter of about 1.0 μm to about 2 μm.

In one embodiment, the diameter of the liposomes is controlled by varying the pore sizes of membranes used in the preparation of the liposomes by extrusion.

In one embodiment, the liposomes comprise between about 1:1 to about 2:1 cholesterol:sphingomyelin (mol %) and have a diameter of about 1.0 μm to about 2 μm.

In another embodiment, the liposomes are multilamellar, comprise between about 1:1 to about 2:1 cholesterol:sphingomyelin (mol %) and have a diameter of about 1.0 μm to about 2 μm.

In one embodiment, the liposomes consist of between about 1:1 to about 2:1 cholesterol:sphingomyelin (mol. %) and have a diameter of about 1.0 μm to about 2 μm.

In another embodiment, the liposomes are multilamellar, consist of between about 1:1 to about 2:1 cholesterol:sphingomyelin (mol. %) and have a diameter of about 1.0 μm to about 2 μm.

In another embodiment, the liposomes are unilamellar, consist of between about 1:1 to about 2:1 cholesterol:sphingomyelin (mol. %) and have a diameter of about 1.0 μm to about 2 μm.

In one embodiment, the liposomes comprise a fluorophore, for instance, fluorescein.

Liposomes of the present invention can be prepared by methods that are known in the art. See, for example, Liposomes: Methods and Protocols, Volume 1: Pharmaceutical Nanocarriers: Methods and Protocols, (ed. Weissig). Humana Press, 2009. ISBN 160327359X; Liposome Technology, volumes I, II & Ill. (ed. Gregoriadis) Informa Healthcare, 2006; and Functional Polymer Colloids and Microparticles volume 4 (Microspheres, microcapsules & iposomes). (eds. Arshady & Guyot). Citus Books, 2002.

Examples of methods suitable for making liposomes of the present invention include extrusion, reverse phase evaporation, sonication, solvent (e.g., ethanol) injection, microfluidization, detergent dialysis, ether injection, and dehydration/rehydration. For instance, one procedure involves dissolving a mixture of at least the liposome-forming lipids, and additionally any other lipids required, in a suitable organic solvent and evaporating the organic solvent in a vessel to form a thin film. The film is then converted to the liposomes by rehydrating with an aqueous medium.

The size of liposomes can be controlled by controlling the pore size of membranes used for low pressure extrusions or the pressure and number of passes utilized in microfluidisation or any other suitable methods.

### Liposome-Toxin Conjugate

As used herein, the term “liposome-toxin conjugate” refers to a conjugate formed on binding of a liposome to a bacterial toxin.

In one embodiment, the liposome is as defined in any of the embodiments of the “liposome” section above.

In one embodiment, the bacterial toxin is as defined in any of the embodiments of the “bacterial toxin” section above.

In one embodiment, the present invention relates to a conjugate comprising:
(i) a liposome comprising/consisting essentially of/consisting of between about 1:1 to about 2:1 cholesterol:sphingomyelin (mol. %) and have a diameter of about 1.0 μm to about 2 μm, and
(ii) a bacterial toxin selected from one of pneumolysin (from Streptococcus pneumoniae), alpha haemolysin (from Staphylococcus aureus and/or Escherichia coli), haemolysin (from Klebsiella pneumoniae), exotoxin A (from Pseudomonas aeruginosa) and streptolysin O (from Streptococcus pyogenes).

In another embodiment, the present invention relates to a conjugate comprising
(i) a liposome comprising/essentially consisting of/consisting of between about 1:1 to about 2:1 cholesterol:sphingomyelin (mol. %) and having a diameter of about 1.0 μm to about 2 μm, and
(ii) a bacterial toxin which is pneumolysin;

Suitably, the binding/conjugation between liposome and toxin may be any commonly encountered means by which chemical/biological entities bind/conjugate to each other, for example, by covalent, ionic, hydrophilic, hydrophobic, Van der Waals, electrostatic and ionic interactions.

### Antibody

The methods of the present invention employ antibodies bound to a label.

In one embodiment, the label bound to the antibody is selected from a fluorochrome label, a radiolabel and a biotin label.

In one embodiment, the label is a fluorochrome label.

As used herein “fluorochrome label” refers to any chemical compound or biological molecule which can emit light of specific intensity and wavelength on excitation, for instance, with light.

Suitable fluorochromes for binding/conjugating antibodies are known in the art. As will be understood, each fluorochromes should be distinguishable by analytic techniques, for example by flow cytometry. Accordingly, when more than one type of antibody is used in the methods of the invention, each type of antibody has a fluorochrome label which is different from the fluorochrome label of the other types of antibody present, such that each type of antibody and the complex they form with a lipsome-toxin conjugate are distinguishable.

The fluorochrome labels are preferably selected for brightness, limited spectral overlap and limited need for compensation, stability, etc.

The following panel of fluorochrome labels is of use: pacific blue (PacB), Horizon V450, pacific orange (PacO), AMCA, AmCyan, fluorescein isothiocyanate (FITC), Alexa488, phycoerythrin (PE), peridinin chlorophyl protein/cyanine 5.5 (PerCP-Cy5.5), PerCP, PE TexasRed, phycoerythrin/cyanine7 (PE-Cy7), allophycocyanine (APC), Alexa647, allophycocyanine/H7 (APC-H7), APC-Cy7, Alexa680 or Alexa700.

In one embodiment, when more than one type of antibody is used, each type of antibody has a label which differs to the label of the other types of antibody present;

In one embodiment, each type of antibody present is bound to one of the fluorochrome labels selected from the numbered groups below. In one embodiment, only one label from each group is present:

(1) pacific blue (PacB) or Horizon V450,
(2) pacific orange (PacO) or AMCA, AmCyan
(3) fluorescein isothiocyanate (FITC) or Alexa488,
(4) phycoerythrin (PE),
(5) peridinin chlorophyl protein/cyanine 5.5 (PerCP-Cy5.5), PerCP or PE TexasRed,
(6) phycoerythrin/cyanine7 (PE-Cy7),
(7) allophycocyanine (APC) or Alexa647, and
(8) allophycocyanine/H7 (APC-H7), APC-Cy7, Alexa680 or Alexa700.

In one embodiment, the liposome-toxin conjugates are incubated with at least one antibody selected from Pneumolysin mouse monoclonal antibody Ply-4 (IgG1 isotype) (available from Abcam® (ab71810)), Lysteriolysin rabbit polyclonal antibody (IgG isotype) (available from Abcam® (ab200538)), Streptolysin mouse monoclonal antibody 6D11 (IgG1 isotype) (available from Abcam® (ab23501)), Alpha hemolysin mouse monoclonal antibody 8B7 (IgG1 isotype) (available from Abcam® (ab190467)), Tetanolysin mouse monoclonal antibody TetE3 (IgG1 isotype) (available from Abcam® (ab64755)), Exotoxin A rabbit polyclonal (from Pseudomonas aeruginosa) N terminal domain I (from whole antiserum) (available from Sigma Aldrich® (P2318)).

Suitably, the liposome-toxin conjugates are incubated with 1, 2, 3, 4, 5 or 6 antibodies selected from Pneumolysin mouse monoclonal antibody Ply-4 (IgG1 isotype) (available from Abcam® (ab71810)), Lysteriolysin rabbit polyclonal antibody (IgG isotype) (available from Abcam® (ab200538)), Streptolysin mouse monoclonal antibody 6D11 (IgG1 isotype) (available from Abcam® (ab23501)), Alpha hemolysin mouse monoclonal antibody 8B7 (IgG1 isotype) (available from Abcam® (ab190467)), Tetanolysin mouse monoclonal antibody TetE3 (IgG1 isotype) (available from Abcam® (ab64755)), Exotoxin A rabbit polyclonal (from Pseudomonas aeruginosa) N terminal domain I (from whole antiserum) (available from Sigma Aldrich® (P2318)).

In one embodiment, the liposome-toxin conjugates are incubated with at least one antibody selected from Pneumolysin mouse monoclonal antibody Ply-4 (IgG1 isotype) (available from Abcam® (ab71810)), Lysteriolysin rabbit polyclonal antibody (IgG isotype) (available from Abcam® (ab200538)), Streptolysin mouse monoclonal antibody 6D11 (IgG1 isotype) (available from Abcam® (ab23501)), Alpha hemolysin mouse monoclonal antibody 8B7 (IgG1 isotype) (available from Abcam® (ab190467)), Tetanolysin mouse monoclonal antibody TetE3 (IgG1 isotype) (available from Abcam® (ab64755)), Exotoxin A rabbit polyclonal (from Pseudomonas aeruginosa) N terminal domain I (from whole antiserum) (available from Sigma Aldrich® (P2318)); and wherein each antibody is labelled with one label selected from pacific blue (PacB), Horizon V450, pacific orange (PacO), AMCA, AmCyan, fluorescein isothiocyanate (FITC), Alexa488, phycoerythrin (PE), peridinin chlorophyl protein/cyanine 5.5 (PerCP-Cy5.5), PerCP, PE TexasRed, phycoerythrin/cyanine7 (PE-Cy7), allophycocyanine (APC), Alexa647, allophycocyanine/H7 (APC-H7), APC-Cy7, Alexa680 or Alexa700.

In one embodiment, the liposome-toxin conjugates are incubated with at least one antibody selected from Pneumolysin mouse monoclonal antibody Ply-4 (IgG1 isotype) (available from Abcam® (ab71810)), Lysteriolysin rabbit polyclonal antibody (IgG isotype) (available from Abcam® (ab200538)), Streptolysin mouse monoclonal antibody 6D11 (IgG1 isotype) (available from Abcam® (ab23501)), Alpha hemolysin mouse monoclonal antibody 8B7 (IgG1 isotype) (available from Abcam® (ab190467)), Tetanolysin mouse monoclonal antibody TetE3 (IgG1 isotype) (available from Abcam® (ab64755)), Exotoxin A rabbit polyclonal (from Pseudomonas aeruginosa) N terminal domain I (from whole antiserum) (available from Sigma Aldrich® (P2318)); and wherein each antibody is labelled with one label selected from pacific blue (PacB), Horizon V450, pacific orange (PacO), AMCA, AmCyan, fluorescein isothiocyanate (FITC), Alexa488, phycoerythrin (PE), peridinin chlorophyl protein/cyanine 5.5 (PerCP-Cy5.5), PerCP, PE TexasRed, phycoerythrin/cyanine7 (PE-Cy7), allophycocyanine (APC), Alexa647, allophycocyanine/H7 (APC-H7), APC-Cy7, Alexa680 or Alexa700; and wherein each type of antibody has a label which differs to the label of the other types of antibody present.

In one embodiment, the liposome-toxin conjugates are incubated with at least one antibody selected from Pneumolysin mouse monoclonal antibody Ply-4 (IgG1 isotype) (available from Abcam® (ab71810)), Lysteriolysin rabbit polyclonal antibody (IgG isotype) (available from Abcam® (ab200538)), Streptolysin mouse monoclonal antibody 6D11 (IgG1 isotype) (available from Abcam® (ab23501)), Alpha hemolysin mouse monoclonal antibody 8B7 (IgG1 isotype) (available from Abcam® (ab190467)), Tetanolysin mouse monoclonal antibody TetE3 (IgG1 isotype) (available from Abcam® (ab64755)), Exotoxin A rabbit polyclonal (from Pseudomonas aeruginosa) N terminal domain I (from whole antiserum) (available from Sigma Aldrich® (P2318)); and wherein each antibody is labelled with one label selected one of the numbered groups below, suitably only one label from each group is present:

(1) pacific blue (PacB) or Horizon V450,
(2) pacific orange (PacO) or AMCA, AmCyan
(3) fluorescein isothiocyanate (FITC) or Alexa488,
(4) phycoerythrin (PE),
(5) peridinin chlorophyl protein/cyanine 5.5 (PerCP-Cy5.5), PerCP or PE TexasRed,
(6) phycoerythrin/cyanine7 (PE-Cy7),
(7) allophycocyanine (APC) or Alexa647, and
(8) allophycocyanine/H7 (APC-H7), APC-Cy7, Alexa680 or Alexa700.

In one embodiment, the liposome-toxin conjugates are incubated with at least one antibody selected from Pneumolysin mouse monoclonal antibody Ply-4 (IgG1 isotype) (available from Abcam® (ab71810)), Lysteriolysin rabbit polyclonal antibody (IgG isotype) (available from Abcam® (ab200538)), Streptolysin mouse monoclonal antibody 6D11 (IgG1 isotype) (available from Abcam® (ab23501)), Alpha hemolysin mouse monoclonal antibody 8B7 (IgG1 isotype) (available from Abcam® (ab190467)), Tetanolysin mouse monoclonal antibody TetE3 (IgG1 isotype) (available from Abcam® (ab64755)), Exotoxin A rabbit polyclonal (from Pseudomonas aeruginosa) N terminal domain I (from whole antiserum) (available from Sigma Aldrich® (P2318)); and wherein each antibody is labelled with one label selected one of the numbered groups below, and wherein each type of antibody has a label which differs to the label of the other types of antibody present, suitably, only one label from each groups is present:

(1) pacific blue (PacB) or Horizon V450,
(2) pacific orange (PacO) or AMCA, AmCyan
(3) fluorescein isothiocyanate (FITC) or Alexa488,
(4) phycoerythrin (PE),
(5) peridinin chlorophyl protein/cyanine 5.5 (PerCP-Cy5.5), PerCP or PE TexasRed,
(6) phycoerythrin/cyanine7 (PE-Cy7),
(7) allophycocyanine (APC) or Alexa647, and
(8) allophycocyanine/H7 (APC-H7), APC-Cy7, Alexa680 or Alexa700.

Methods of antibody labelling are well known in the art. Antibodies may be directly or indirectly linked. In one embodiment, antibodies are directly linked.

Fluorochrome labelled antibodies may be prepared using a commercially available labelling kits, such as Lightening Link® products.

### Conjugate-Antibody Complex

As used herein the term “conjugate-antibody complex” refers to the complex formed when the labelled antibody binds to the liposome-toxin conjugate. Suitably, the labelled antibody is a fluorochrome labelled antibody.

Suitably, the binding between labelled antibody and liposome-toxin conjugate may be any commonly encountered means by which chemical/biological entities bind to each other, for example, by covalent, ionic, hydrophilic, hydrophobic, Van der Waals, electrostatic and ionic interactions.

In one aspect, the present invention relates to a complex comprising:

(i) a conjugate comprising a lipsome and a bacterial toxin; and

(ii) an antibody bound to a label.

In another aspect, the present invention relates to a complex comprising:

(iii) a conjugate comprising a liposome and a bacterial toxin; and

(iv) an antibody bound to a fluorochrome label.

In one embodiment, the liposome may be as defined in any of the embodiments.

In one embodiment, the bacterial toxin may be as defined in any of the embodiments above.

In one embodiment, the antibody may be as defined in any of the embodiments above.

In one embodiment, the label may be as defined in any of the embodiments above.

In one embodiment, the antibody bound to a label may be as defined in any of the embodiments above.

In one embodiment, the present invention relates to a complex comprising:
(i) a conjugate comprising a liposome and a bacterial toxin; wherein the liposome consists of between about 1:1 to about 2:1 cholesterol:sphingomyelin (mol. %) and have a diameter of about 1.0 μm to about 2 μm, and wherein the bacterial toxin is selected from one of pneumolysin (from Streptococcus pneumoniae), alpha haemolysin (from Staphylococcus aureus and/or Escherichia coli), haemolysin (from Klebsiella pneumoniae), exotoxin A (from Pseudomonas aeruginosa) and streptolysin O (from Streptococcus pyogenes); and.
(ii) a labelled antibody.

In another embodiment, the present invention relates to a complex comprising:
(i) a conjugate comprising a liposome and a bacterial toxin; wherein the lipsome consists of between about 1:1 to about 2:1 cholesterol:sphingomyelin (mol. %) and have a diameter of about 1.0 μm to about 2 μm, and wherein the bacterial toxin is selected from one of pneumolysin (from Streptococcus pneumoniae), alpha haemolysin (from Staphylococcus aureus and/or Escherichia coli), haemolysin (from Klebsiella pneumoniae), exotoxin A (from Pseudomonas aeruginosa) and streptolysin O (from Streptococcus pyogenes); and.
(ii) a fluorochrome labelled antibody.

In another embodiment, the present invention relates to a complex comprising:
(i) a conjugate comprising a liposome and a bacterial toxin; wherein the liposome consists of between about 1:1 to about 2:1 cholesterol:sphingomyelin (mol. %) and have a diameter of about 1.0 μm to about 2 μm, and wherein the bacterial toxin is pneumolysin; and
(ii) a fluorochrome labelled antibody which is mouse monoclonal antibody Ply-4 (IgG1 isotype) available from Abcam® (ab71810) labelled with APC.

In another embodiment, the present invention relates to a complex comprising:
(i) a conjugate comprising a liposome and a bacterial toxin; wherein the liposome consists of between about 1:1 to about 2:1 cholesterol:sphingomyelin (mol. %) and have a diameter of about 1.0 μm to about 2 μm, and wherein the bacterial toxin is pneumolysin; and
(ii) a fluorochrome labelled antibody, wherein the antibody is selected from Pneumolysin mouse monoclonal antibody Ply-4 (IgG1 isotype) (available from Abcam® (ab71810)), Lysteriolysin rabbit polyclonal antibody (IgG isotype) (available from Abcam® (ab200538)), Streptolysin mouse monoclonal antibody 6D11 (IgG1 isotype) (available from Abcam® (ab23501)), Alpha hemolysin mouse monoclonal antibody 8B7 (IgG1 isotype) (available from Abcam® (ab190467)), Tetanolysin mouse monoclonal antibody TetE3 (IgG1 isotype) (available from Abcam® (ab64755)), Exotoxin A rabbit polyclonal (from Pseudomonas aeruginosa) N terminal domain I (from whole antiserum) (available from Sigma Aldrich® (P2318)) and the label is selected from pacific blue (PacB), Horizon V450, pacific orange (PacO), AMCA, AmCyan, fluorescein isothiocyanate (FITC), Alexa488, phycoerythrin (PE), peridinin chlorophyl protein/cyanine 5.5 (PerCP-Cy5.5), PerCP, PE TexasRed, phycoerythrin/cyanine7 (PE-Cy7), allophycocyanine (APC), Alexa647, allophycocyanine/H7 (APC-H7), APC-Cy7, Alexa680 or Alexa700.

### Method

In one aspect the present invention relates to a method for detecting the presence of one or more bacterial toxins, capable of binding to cell membranes, in biological fluid wherein the method comprises:

(i) incubating the biological fluid with a plurality of liposomes, wherein the liposomes comprise a lipid capable of binding to said one or more toxins, to provide one or more liposome-toxin conjugate(s);
(ii) incubating said conjugate(s) with at least one type of antibody bound to a label to provide one or more conjugate-antibody complex(es);

wherein each type of antibody in the mixture is specific for one of the bacterial toxins whose presence is to be detected; and

(iii) analysing said complex(es) in order to detect the presence of one or more bacterial toxins capable of binding to cell membranes.

In another aspect the present invention relates to a method for the diagnosis of sepsis wherein the method comprises:

(i) incubating a biological sample from a patient suspected of suffering from sepsis with a plurality of liposomes, wherein the liposomes comprise a lipid capable of binding to one or more bacterial toxin produced by bacteria implicated in the development of sepsis, to provide one or more liposome-toxin conjugate(s);
(ii) incubating said conjugates with at least one type of antibody bound to a label to provide one or more conjugate-antibody complex(es);

wherein each type of antibody in the mixture is specific for a bacterial toxin produced by bacteria implicated in the development of sepsis; and

(iii) analysing said complex(es) in order to detect the presence of bacterial toxins produced by bacteria implicated in the development of sepsis.

In another aspect, the present invention relates to a method for determining the prognosis of a patient suspected of suffering from sepsis wherein the method comprises:

(i) incubating a biological sample from a patient suspected of suffering from sepsis with a plurality of liposomes, wherein the liposomes comprise a lipid capable of binding to one or more bacterial toxin produced by bacteria implicated in the development of sepsis, to provide one or more liposome-toxin conjugate(s);
(ii) incubating said conjugate(s) with at least one type of antibody bound to a label to provide one or more conjugate-antibody complex(es);

wherein each type of antibody in the mixture is specific for a bacterial toxin produced by bacteria implicated in the development of sepsis; and

(iii) analysing said complex(es) in order to detect quantitatively the presence of bacterial toxins produced by bacteria implicated in the development of sepsis.

In one embodiment, the present invention relates to a method for detecting the presence of one or more bacterial toxins, capable of binding to cell membranes, in biological fluid wherein the method comprises:

(i) incubating the biological fluid with a plurality of liposomes, wherein the liposomes comprise a lipid capable of binding to said one or more toxins, to provide one or more liposome-toxin conjugate(s);
(ii) incubating said conjugate(s) with at least one type of antibody bound to a label to provide one or more conjugate-antibody complex(es);

wherein each type of antibody in the mixture is specific for one of the bacterial toxins whose presence is to be detected; and

wherein, when more than one type of antibody is used, each type of antibody has a label which differs to the label of the other types of antibody present;

(iii) analysing said complex(es) in order to detect the presence of one or more bacterial toxins capable of binding to cell membranes.

In another embodiment, the present invention relates to a method for the diagnosis of sepsis wherein the method comprises:

(i) incubating a biological sample from a patient suspected of suffering from sepsis with a plurality of liposomes, wherein the liposomes comprise a lipid capable of binding to one or more bacterial toxin produced by bacteria implicated in the development of sepsis, to provide one or more liposome-toxin conjugate(s);
(ii) incubating said conjugates with at least one type of antibody bound to a label to provide one or more conjugate-antibody complex(es);

wherein each type of antibody in the mixture is specific for a bacterial toxin produced by bacteria implicated in the development of sepsis; and

wherein, when more than one type of antibody is used, each type of antibody has a label which differs to the label of the other types of antibody present;

(iii) analysing said complex(es) in order to detect the presence of bacterial toxins produced by bacteria implicated in the development of sepsis.

In another embodiment, the present invention relates to a method for determining the prognosis of a patient suspected of suffering from sepsis wherein the method comprises:

(i) incubating a biological sample from a patient suspected of suffering from sepsis with a plurality of liposomes, wherein the liposomes comprise a lipid capable of binding to one or more bacterial toxin produced by bacteria implicated in the development of sepsis, to provide one or more liposome-toxin conjugate(s);
(ii) incubating said conjugate(s) with at least one type of antibody bound to a label to provide one or more conjugate-antibody complex(es);

wherein each type of antibody in the mixture is specific for a bacterial toxin produced by bacteria implicated in the development of sepsis; and

wherein, when more than one type of antibody is used, each type of antibody has a label which differs to the label of the other types of antibody present;

(iii) analysing said complex(es) in order to detect quantitatively the presence of bacterial toxins produced by bacteria implicated in the development of sepsis.

In each of these aspects the biological sample, bacterial toxin, liposomes, antibody, and label may be as described in the relevant sections above.

In each of the above aspects, in one embodiment, the label bound to the antibody is a fluorochrome label.

In one embodiment, each of the above methods are in vitro methods.

In one embodiment, the biological sample and liposomes are incubated at a temperature of from about 1° C. to about 37° C. Suitably, the biological sample and liposomes are incubated at a temperature of about 1° C. to about 25° C. Suitably, the biological sample and liposomes are incubated at a temperature of about 1° C. to about 10° C. Suitably, the biological sample and liposomes are incubated at a temperature of about 4° C.

In one embodiment, the biological sample and liposome are incubated for between about 1 and about 45 minutes. Suitably, the biological sample and liposomes are incubated for about 30 minutes.

In one embodiment, between about 0.1 to 100 μg/ml of liposomes are incubated with the biological sample. Suitably, between about 0.1 to 50 μg/ml of liposomes are incubated with the biological sample. Suitably, between about 0.1 to 20 μg/ml of liposomes are incubated with the biological sample. Suitably, between about 0.1 to 10 μg/ml of liposomes are incubated with the biological sample. Suitably, about 1 μg/ml of liposomes are incubated with the biological sample.

In one embodiment, the biological sample is incubated with about 1 μg/ml of liposomes for about 30 minutes at about 4° C.

In one embodiment, the liposome-toxin conjugate and at least one antibody bound to a label are incubated at a temperature of about 1° C. to about 37° C. Suitably, the conjugate and antibody are incubated at a temperature of about 1° C. to about 25° C. Suitably, the conjugate and antibody are incubated at a temperature of about 1° C. to about 10° C. Suitably, liposome-toxin conjugate and at least one antibody bound to a label are incubated at a temperature of about 4° C.

In one embodiment, the liposome-toxin conjugate and at least one antibody bound to a label are incubated in a buffer, suitably a PBS buffer.

In one embodiment, the liposome-toxin conjugate and at least one antibody bound to a label are incubated for between about 1 and about 45 minutes. Suitably, the liposome-toxin conjugate and at least one antibody bound to a label are incubated for about 30 minutes.

In one embodiment, the liposome-toxin conjugate and at least one antibody bound to a label are incubated for about 30 minutes at about 4° C.

In one embodiment, the present invention provides a method for detecting the presence of one or more bacterial toxins, capable of binding to cell membranes, in biological fluid wherein the method comprises:

(i) incubating the biological fluid with a plurality of liposomes, wherein the liposomes comprise a lipid capable of binding to said one or more toxins, to provide one or more liposome-toxin conjugate(s);
(ii) isolating the one or more liposome-toxin conjugate(s);
(iii) incubating said conjugate(s) with at least one type of antibody bound to a label to provide one or more conjugate-antibody complex(es);

wherein each type of antibody in the mixture is specific for one of the bacterial toxins whose presence is to be detected; and

(iv) isolating said one or more conjugate-antibody complex(es); and
(v) analysing said complex(es) in order to detect the presence of one or more bacterial toxins capable of binding to cell membranes.

In one embodiment, the present invention provides a method for detecting the presence of one or more bacterial toxins, capable of binding to cell membranes, in biological fluid wherein the method comprises:

(i) incubating the biological fluid with a plurality of liposomes, wherein the liposomes comprise a lipid capable of binding to said one or more toxins, to provide one or more liposome-toxin conjugate(s);
(ii) isolating the one or more liposome-toxin conjugate(s);
(iii) incubating said conjugate(s) with at least one type of antibody bound to a label to provide one or more conjugate-antibody complex(es);

wherein each type of antibody in the mixture is specific for one of the bacterial toxins whose presence is to be detected; and

wherein, when more than one type of antibody is used, each type of antibody has a label which differs to the label of the other types of antibody present;

(iv) isolating said one or more conjugate-antibody complex(es); and
(v) analysing said complex(es) in order to detect the presence of one or more bacterial toxins capable of binding to cell membranes.

In another embodiment, the present invention relates to a method for the diagnosis of sepsis wherein the method comprises:

(i) incubating a biological sample from a patient suspected of suffering from sepsis with a plurality of liposomes, wherein the liposomes comprise a lipid capable of binding to one or more bacterial toxin produced by bacteria implicated in the development of sepsis, to provide one or more liposome-toxin conjugate(s);
(ii) isolating the one or more liposome-toxin conjugate(s);
(iii) incubating said conjugate(s) with at least one type of antibody bound to a label to provide one or more conjugate-antibody complex(es);

wherein each type of antibody in the mixture is specific for a bacterial toxin produced by bacteria implicated in the development of sepsis; and

(iv) isolating said one or more conjugate-antibody complex(es); and
(v) analysing said complex(es) in order to detect the presence of bacterial toxins produced by bacteria implicated in the development of sepsis.

In another embodiment, the present invention relates to a method for the diagnosis of sepsis wherein the method comprises:

(i) incubating a biological sample from a patient suspected of suffering from sepsis with a plurality of liposomes, wherein the liposomes comprise a lipid capable of binding to one or more bacterial toxin produced by bacteria implicated in the development of sepsis, to provide one or more liposome-toxin conjugate(s);
(ii) isolating the one or more liposome-toxin conjugate(s);
(iii) incubating said conjugate(s) with at least one type of antibody bound to a label to provide one or more conjugate-antibody complex(es);

wherein each type of antibody in the mixture is specific for a bacterial toxin produced by bacteria implicated in the development of sepsis; and

wherein, when more than one type of antibody is used, each type of antibody has a label which differs to the label of the other types of antibody present;

(iv) isolating said one or more conjugate-antibody complex(es); and
(v) analysing said complex(es) in order to detect the presence of bacterial toxins produced by bacteria implicated in the development of sepsis.

In another embodiment, the present invention relates to a method for determining the prognosis of a patient suspected of suffering from sepsis wherein the method comprises:

(i) incubating a biological sample from a patient suspected of suffering from sepsis with a plurality of liposomes, wherein the liposomes comprise a lipid capable of binding to one or more bacterial toxin produced by bacteria implicated in the development of sepsis, to provide one or more liposome-toxin conjugate(s);
(ii) isolating the one or more liposome-toxin conjugate(s);
(iii) incubating said conjugate(s) with at least one type of antibody bound to a label to provide one or more conjugate-antibody complex(es);

wherein each type of antibody in the mixture is specific for a bacterial toxin produced by bacteria implicated in the development of sepsis; and

(iv) isolating said one or more conjugate-antibody complex(es); and
(v) analysing said complexes in order to detect quantitatively the presence of bacterial toxins produced by bacteria implicated in the development of sepsis.

In another embodiment, the present invention relates to a method for determining the prognosis of a patient suspected of suffering from sepsis wherein the method comprises:

(i) incubating a biological sample from a patient suspected of suffering from sepsis with a plurality of liposomes, wherein the liposomes comprise a lipid capable of binding to one or more bacterial toxin produced by bacteria implicated in the development of sepsis, to provide one or more liposome-toxin conjugate(s);
(ii) isolating the one or more liposome-toxin conjugate(s);
(iii) incubating said conjugate(s) with at least one type of antibody bound to a label to provide one or more conjugate-antibody complex(es);

wherein each type of antibody in the mixture is specific for a bacterial toxin produced by bacteria implicated in the development of sepsis; and

wherein, when more than one type of antibody is used, each type antibody has a label which differs to the label of the other types of antibody present;

(iv) isolating said one or more conjugate-antibody complex(es); and
(v) analysing said complexes in order to detect quantitatively the presence of bacterial toxins produced by bacteria implicated in the development of sepsis.

In each of the above embodiments, the conjugates and/or complexes may be isolated by techniques commonly known in the art. For example, the one or more liposome-toxin conjugate(s) and conjugate-antibody complex(es) may be isolated by centrifugation.

In each of the above embodiments, the label bound to the antibody is suitably a fluorochrome label.

Suitably, centrifugation may be carried out at between about 1000 to about 14000×g for about 1 to about 30 minutes. Suitably, centrifugation may be carried out at about 13,000 to 14,000×g for about 10 minutes.

In each of the above embodiments, the conjugate-antibody complexes may be analysed by any suitable method known in the art. For example, flow cytometry, enzyme-linked immunosorbent assay, mass spectrometry and nuclear magnetic.

Advantageously, the conjugate-antibody complexes may be analysed by flow cytometry. Suitably, multi-colour flow cytometry. In one embodiment, multicolour flow cytometry is used with at least 2 fluorescence detection channels, suitably at least 4 fluorescence detection channels, suitably at least 6 fluorescence detection channels, suitably at least 8 fluorescence detection channels.

Flow cytometry techniques would be familiar to a person skilled in the art. Further, flow cytometry methods are described in Handbook of Flow Cytometry Method, J. Paul Robinson (Editor); Flow Cytometry—A Basic Introduction, Michael G Ormerod (2008) and Current Protocols in Cytometry (2010), Wiley.

In one embodiment, the results of the analysis are compared to data generated using known concentrations of bacterial toxin in order to quantify the presence of bacterial toxin. The skilled person would understand that this information can be used to assess patient prognosis.

### Kit

In another aspect, the present invention relates to a kit for detecting the presence of bacterial toxins comprising:

(i) a container comprising liposomes and optionally a buffer, wherein the liposomes comprise a lipid capable of binding to one or more bacterial toxins; and
(ii) a container comprising a reagent, wherein the reagent comprises at least one type of antibody bound to a label; and
(iii) optionally further containers comprising a reagent, wherein the reagent comprises at least one type of antibody bound to a label;
wherein each type of antibody in the kit is specific for a different bacterial toxin to be detected.

In one embodiment, when more than one type of antibody is present in the kit, each type antibody has a label which differs to the label of the other types of antibody present in the kit.

In one embodiment, the label bound to the antibody is a fluorochrome label.

In one embodiment, the liposome may be as defined in any of the embodiments.

In one embodiment, the bacterial toxin may be as defined in any of the embodiments above.

In one embodiment, the antibody may be as defined in any of the embodiments above.

In one embodiment, the label may be as defined in any of the embodiments above.

In one embodiment, the antibody bound to a label may be as defined in any of the embodiments above.

The invention will now be further described by way of the following numbered paragraphs:

1. A method for detecting the presence of one or more bacterial toxins, capable of binding to cell membranes, in biological fluid wherein the method comprises:
(i) incubating the biological fluid with a plurality of liposomes, wherein the liposomes comprise a lipid capable of binding to said one or more toxins, to provide one or more liposome-toxin conjugate(s);
(ii) incubating said conjugates with at least one type of antibody bound to a label to provide one or more conjugate-antibody complex(es);

wherein each type of antibody in the mixture is specific for one of the bacterial toxins whose presence is to be detected; and

(iii) analysing said complex(es) in order to detect the presence of one or more bacterial toxins capable of binding to cell membranes.
2. A method according to paragraph 1 wherein the method is an in vitro method.
3. A method according to any preceding paragraph wherein the biological fluid is human biological fluid.
4. A method according to any preceding paragraph wherein the biological fluid is an ex vivo sample of biological fluid.
5. A method according to any preceding paragraph wherein the biological fluid is selected from one of whole blood, blood plasma, blood serum, CSF or urine.
6. A method according to any preceding paragraph wherein the one or more bacterial toxins are bacterial toxins capable of binding to eukaryotic cell membranes.
7. A method according to any preceding paragraph wherein the one or more bacterial toxins are bacterial toxins capable of binding to mammalian cell membranes.
8. A method according to any preceding paragraph wherein the one or more bacterial toxins are bacterial toxins capable of binding to human cell membranes.
9. A method according to any preceding paragraph wherein the one or more bacterial toxins are involved in the aetiology of one or more of sepsis, pneumonia, meningitis and urinary tract infections.
10. A method according to any preceding paragraph wherein the one or more bacterial toxins are involved in the aetiology of sepsis.
11. A method according to any preceding paragraph wherein the one or more bacterial toxins are derived from one or more of Streptococcus pneumoniae, Staphylococcus aureus, Escherichia coli, Klebsiella pneumoniae, Pseudomonas aeruginosa and Streptococcus pyogenes.
12. A method according to any preceding paragraph wherein the one or more bacterial toxins are selected from pneumolysin (from Streptococcus pneumoniae), alpha haemolysin (from Staphylococcus aureus and/or Escherichia coli), haemolysin (from Klebsiella pneumoniae), exotoxin A (from Pseudomonas aeruginosa) and streptolysin O (from Streptococcus pyogenes).
13. A method according to any preceding paragraph wherein the one or more bacterial toxins are a combination of pneumolysin (from Streptococcus pneumoniae), alpha haemolysin (from Staphylococcus aureus and/or Escherichia coli), haemolysin (from Klebsiella pneumoniae), exotoxin A (from Pseudomonas aeruginosa) and streptolysin O (from Streptococcus pyogenes).
14. A method according to any preceding paragraph wherein the liposomes comprise cholesterol.
15. A method according to any preceding paragraph wherein the liposome comprise at least about 20 mol. %, preferably at least about 25 mol. %, more preferably at least about 30 mol. %, more preferably at least about 50 mol. % of cholesterol.
16. A method according to any preceding paragraph wherein the liposomes comprise at least about 20 mol. % to about 66 mol. % of cholesterol, preferably at least about 25 mol. % to about 66 mol. % of cholesterol, more preferably at least about 30 mol. % to about 66 mol. % of cholesterol, more preferably at least about 50 mol. % to about 66 mol. % of cholesterol.
17. A method according to any preceding paragraph wherein the liposomes comprise cholesterol and sphingomyelin.
18. A method according to any preceding paragraph wherein the liposomes consist essentially of cholesterol and sphingomyelin.
19. A method according to any preceding paragraph wherein the liposomes consist of cholesterol and sphingomyelin.
20. A method according to any preceding paragraph wherein the ratio of cholesterol t sphingomyelin is about 1:2 to about 2:1 cholesterol:sphingomyelin (mol %), preferably about 1:1 to about 2:1 cholesterol:sphingomyelin (mol %).
21. A method according to any preceding paragraph wherein the liposomes are multilamellar.
22. A method according to any preceding clam wherein the liposome are unilamellar.
23. A method according to any preceding paragraph wherein the liposomes are at least about 0.4 μm in diameter.
24. A method according to any preceding paragraph wherein the liposomes have a diameter of about 0.4 μm to about 2 μm.
25. A method according to any preceding paragraph wherein the liposomes have a diameter of about 1.0 μm to about 2 μm.
26. A method according to any preceding paragraph wherein the incubation of step (i) is performed at a temperature of about 1° C. to about 37° C.
27. A method according to any preceding paragraph wherein the incubation of step (ii) is performed at a temperature of about 1° C. to about 37° C.
28. A method according to any preceding paragraph wherein the antibodies are selected from at least one of Pneumolysin mouse monoclonal antibody Ply-4 (IgG1 isotype) (available from Abcam® (ab71810)), Lysteriolysin rabbit polyclonal antibody (IgG isotype) (available from Abcam® (ab200538)), Streptolysin mouse monoclonal antibody 6D11 (IgG1 isotype) (available from Abcam® (ab23501)), Alpha hemolysin mouse monoclonal antibody 8B7 (IgG1 isotype) (available from Abcam® (ab190467)), Tetanolysin mouse monoclonal antibody TetE3 (IgG1 isotype) (available from Abcam® (ab64755)), Exotoxin A rabbit polyclonal (from Pseudomonas aeruginosa) N terminal domain I (from whole antiserum) (available from Sigma Aldrich® (P2318)).
29. A method according to any preceding paragraph wherein the label bound to the antibody is a fluorochrome label.
30. A method according to any preceding paragraph wherein the label is selected from pacific blue (PacB), Horizon V450, pacific orange (PacO), AMCA, AmCyan, fluorescein isothiocyanate (FITC), Alexa488, phycoerythrin (PE), peridinin chlorophyl protein/cyanine 5.5 (PerCP-Cy5.5), PerCP, PE TexasRed, phycoerythrin/cyanine7 (PE-Cy7), allophycocyanine (APC), Alexa647, allophycocyanine/H7 (APC-H7), APC-Cy7, Alexa680 or Alexa700.
31. A method according to any preceding paragraph wherein when more than one type of antibody is used, each type of antibody has a label which differs to the label of the other types of antibody present.
32. A method according to any preceding paragraph wherein the bacterial toxins comprises pneumolysin and the antibody specific for pneumolysin is Ply-4 (IgG1 isotype).
33. A method according to any preceding paragraph wherein the bacterial toxins comprise pneumolysin and the antibody specific for pneumolysin is Ply-4 (IgG1 isotype) conjugated to allophycocyanin.
34. A method according to any preceding paragraph wherein the analysis of step (iii) is performed using flow cytometry.
35. A method according to any preceding paragraph wherein the analysis of step (iii) is performed using multi-colour flow cytometry.
36. A method for the diagnosis of sepsis wherein the method comprises:
(i) incubating a biological sample from a patient suspected of suffering from sepsis with a plurality of liposomes, wherein the liposomes comprise a lipid capable of binding to one or more bacterial toxin produced by bacteria implicated in the development of sepsis, to provide one or more liposome-toxin conjugate(s);
(ii) incubating said conjugate(s) with at least one type of antibody bound to a label to provide one or more conjugate-antibody complex(es);

wherein each type of antibody in the mixture is specific for a bacterial toxin produced by bacteria implicated in the development of sepsis; and

(iii) analysing said complex(es) in order to detect the presence of bacterial toxins produced by bacteria implicated in the development of sepsis.
37. A method according to paragraph 36 wherein the method is an in vitro method.
38. A method according to any one of paragraphs 36 to 37 wherein the biological fluid is human biological fluid.
39. A method according to any one of paragraphs 36 to 38 wherein the biological fluid is an ex vivo sample of biological fluid.
40. A method according to any one of paragraphs 36 to 39 wherein the biological fluid is selected from one of whole blood, blood plasma, blood serum, CSF or urine.
41. A method according to any one of paragraphs 36 to 40 wherein the at least one bacterial toxin is derived from one or more of Streptococcus pneumoniae, Staphylococcus aureus, Escherichia coli, Klebsiella pneumoniae, Pseudomonas aeruginosa and Streptococcus pyogenes.
42. A method according to any one of paragraphs 36 to 41 wherein the at least one bacterial toxin is selected from pneumolysin (from Streptococcus pneumoniae), alpha haemolysin (from Staphylococcus aureus and/or Escherichia coli), haemolysin (from Klebsiella pneumoniae), exotoxin A (from Pseudomonas aeruginosa) and streptolysin O (from Streptococcus pyogenes).
43. A method according to any one of paragraphs 36 to 42 wherein the at least one bacterial toxin is a mixture of pneumolysin (from Streptococcus pneumoniae), alpha haemolysin (from Staphylococcus aureus and/or Escherichia coli), haemolysin (from Klebsiella pneumoniae), exotoxin A (from Pseudomonas aeruginosa) and streptolysin O (from Streptococcus pyogenes).
44. A method according to any one of paragraphs 36 to 43 wherein the liposomes comprise cholesterol.
45. A method according to any one of paragraphs 36 to 44 wherein the liposome comprise at least about 20 mol. %, preferably at least about 25 mol. %, more preferably at least about 30 mol. %, more preferably at least about 50 mol. % of cholesterol.
46. A method according to any one of paragraphs 36 to 45 wherein the liposomes comprise at least about 20 mol. % to about 66 mol. % of cholesterol, preferably at least about 25 mol. % to about 66 mol. % of cholesterol, more preferably at least about 30 mol. % to about 66 mol. % of cholesterol, more preferably at least about 50 mol. % to about 66 mol. % of cholesterol.
47. A method according to any one of paragraphs 36 to 46 wherein the liposomes comprise cholesterol and sphingomyelin.
48. A method according to any one of paragraphs 36 to 47 wherein the liposomes consist essentially of cholesterol and sphingomyelin.
49. A method according to any one of paragraphs 36 to 48 wherein the liposomes consist of cholesterol and sphingomyelin.
50. A method according to any one of paragraphs 36 to 49 wherein the ratio of cholesterol t sphingomyelin is about 1:2 to about 2:1 cholesterol:sphingomyelin (mol %), preferably about 1:1 to about 2:1 cholesterol:sphingomyelin (mol %).
51. A method according to any one of paragraphs 36 to 50 wherein the liposomes are multilamellar.
52. A method according to any one of paragraphs 36 to 51 wherein the liposome are unilamellar.
53. A method according to any one of paragraphs 36 to 52 wherein the liposome are at least about 0.4 μm in diameter.
54. A method according to any one of paragraphs 36 to 53 wherein the liposomes have a diameter of about 0.4 μm to about 2 μm.
55. A method according to any one of paragraphs 36 to 54 wherein the liposomes have a diameter of about 1.0 μm to about 2 μm.
56. A method according to any one of paragraphs 36 to 55 wherein the incubation of step (i) is performed at a temperature of about 1° C. to about 37° C.
57. A method according to any one of paragraphs 36 to 56 wherein the incubation of step (ii) is performed at a temperature of about 1° C. to about 37° C.
58. A method according to any one of paragraphs 36 to 57 wherein the antibodies are selected from Pneumolysin mouse monoclonal antibody Ply-4 (IgG1 isotype) (available from Abcam® (ab71810)), Lysteriolysin rabbit polyclonal antibody (IgG isotype) (available from Abcam® (ab200538)), Streptolysin mouse monoclonal antibody 6D11 (IgG1 isotype) (available from Abcam® (ab23501)), Alpha hemolysin mouse monoclonal antibody 8B7 (IgG1 isotype) (available from Abcam® (ab190467)), Tetanolysin mouse monoclonal antibody TetE3 (IgG1 isotype) (available from Abcam® (ab64755)), Exotoxin A rabbit polyclonal (from Pseudomonas aeruginosa) N terminal domain I (from whole antiserum) (available from Sigma Aldrich® (P2318)).
59. A method according to any one of paragraphs 36 to 58 wherein the label bound to the antibody is a fluorochrome label.
60. A method according any one of paragraphs 36 to 59 wherein the label is selected from pacific blue (PacB), Horizon V450, pacific orange (PacO), AMCA, AmCyan, fluorescein isothiocyanate (FITC), Alexa488, phycoerythrin (PE), peridinin chlorophyl protein/cyanine 5.5 (PerCP-Cy5.5), PerCP, PE TexasRed, phycoerythrin/cyanine7 (PE-Cy7), allophycocyanine (APC), Alexa647, allophycocyanine/H7 (APC-H7), APC-Cy7, Alexa680 or Alexa700.
61. A method according to any one of paragraphs 36 to 60 wherein when more than one type of antibody is used, each type of antibody has a label which differs to the label of the other types of antibody present;
62. A method according to any one of paragraphs 36 to 61 wherein the bacterial toxin comprises pneumolysin and the antibody specific for pneumolysin is Ply-4 (IgG1 isotype).
63. A method according to any one of paragraphs 36 to 62 wherein the bacterial toxin comprises pneumolysin and the antibody specific for pneumolysin is Ply-4 (IgG1 isotype) conjugated to allophycocyanin.
64. A method according to any one of paragraphs 36 to 63 wherein the analysis of step (iii) is performed using flow cytometry.
65. A method according to any one of paragraphs 36 to 64 wherein the analysis of step (iii) is performed using multi-colour flow cytometry.
66. A method for determining the prognosis of a patient suspected of suffering from sepsis wherein the method comprises:
(i) incubating a biological sample from a patient suspected of suffering from sepsis with a plurality of liposomes, wherein the liposomes comprise a lipid capable of binding to one or more bacterial toxin produced by bacteria implicated in the development of sepsis, to provide one or more liposome-toxin conjugate(s);
(ii) incubating said conjugate(s) with at least one type of antibody bound to a label to provide one or more conjugate-antibody complex(es);

wherein each type of antibody in the mixture is specific for a bacterial toxin produced by bacteria implicated in the development of sepsis; and

(iii) analysing said complex(es) in order to detect quantitatively the presence of bacterial toxins produced by bacteria implicated in the development of sepsis.
67. A method according to paragraph 66 wherein the method is an in vitro method.
68. A method according to any one of paragraphs 66 to 67 wherein the biological fluid is human biological fluid.
69. A method according to any one of paragraphs 66 to 68 wherein the biological fluid is an ex vivo sample of biological fluid.
70. A method according to any one of paragraphs 66 to 69 wherein the biological fluid is selected from one of whole blood, blood plasma, blood serum, CSF or urine.
71. A method according to any one of paragraphs 66 to 70 wherein the at least one bacterial toxin is derived from one or more of Streptococcus pneumoniae, Staphylococcus aureus, Escherichia coli, Klebsiella pneumoniae, Pseudomonas aeruginosa and Streptococcus pyogenes.
72. A method according to any one of paragraphs 66 to 71 wherein the at least one bacterial toxin is selected from pneumolysin (from Streptococcus pneumoniae), alpha haemolysin (from Staphylococcus aureus and/or Escherichia coli), haemolysin (from Klebsiella pneumoniae), exotoxin A (from Pseudomonas aeruginosa) and streptolysin O (from Streptococcus pyogenes).
73. A method according to any one of paragraphs 66 to 72 wherein the at least one bacterial toxins are pneumolysin (from Streptococcus pneumoniae), alpha haemolysin (from Staphylococcus aureus and/or Escherichia coli), haemolysin (from Klebsiella pneumoniae), exotoxin A (from Pseudomonas aeruginosa) and streptolysin O (from Streptococcus pyogenes).
74. A method according to any one of paragraphs 66 to 73 wherein the liposomes comprise cholesterol.
75. A method according to any one of paragraphs 66 to 74 wherein the liposome comprise at least about 20 mol. %, preferably at least about 25 mol. %, more preferably at least about 30 mol. %, more preferably at least about 50 mol. % of cholesterol.
76. A method according to any one of paragraphs 66 to 75 wherein the liposomes comprise at least about 20 mol. % to about 66 mol. % of cholesterol, preferably at least about 25 mol. % to about 66 mol. % of cholesterol, more preferably at least about 30 mol. % to about 66 mol. % of cholesterol, more preferably at least about 50 mol. % to about 66 mol. % of cholesterol.
77. A method according to any one of paragraphs 66 to 76 wherein the liposomes comprise cholesterol and sphingomyelin.
78. A method according to any one of paragraphs 66 to 77 wherein the liposomes consist essentially of cholesterol and sphingomyelin.
79. A method according to any one of paragraphs 66 to 78 wherein the liposomes consist of cholesterol and sphingomyelin.
80. A method according to any one of paragraphs 66 to 79 wherein the ratio of cholesterol t sphingomyelin is about 1:2 to about 2:1 cholesterol:sphingomyelin (mol %), preferably about 1:1 to about 2:1 cholesterol:sphingomyelin (mol %). 81. A method according to any one of paragraphs 66 to 80 wherein the liposomes are multilamellar.
82. A method according to any one of paragraphs 66 to 80 wherein the liposomes are unilamellar.
83. A method according to any one of paragraphs 66 to 82 wherein the liposomes are at least about 0.4 μm in diameter.
84. A method according to any one of paragraphs 66 to 83 wherein the liposomes have a diameter of about 0.4 μm to about 2 μm.
85. A method according to any one of paragraphs 66 to 84 wherein the liposomes have a diameter of about 1.0 μm to about 2 μm.
86. A method according to any one of paragraphs 66 to 85 wherein the incubation of step (i) is performed at a temperature of about 1° C. to about 37° C.
87. A method according to any one of paragraphs 66 to 86 wherein the incubation of step (ii) is performed at a temperature of about 1° C. to about 37° C.
88. A method according to any one of paragraphs 66 to 87 wherein the antibodies are selected from Pneumolysin mouse monoclonal antibody Ply-4 (IgG1 isotype) (available from Abcam® (ab71810)), Lysteriolysin rabbit polyclonal antibody (IgG isotype) (available from Abcam® (ab200538)), Streptolysin mouse monoclonal antibody 6D11 (IgG1 isotype) (available from Abcam® (ab23501)), Alpha hemolysin mouse monoclonal antibody 8B7 (IgG1 isotype) (available from Abcam® (ab190467)), Tetanolysin mouse monoclonal antibody TetE3 (IgG1 isotype) (available from Abcam® (ab64755)), Exotoxin A rabbit polyclonal (from Pseudomonas aeruginosa) N terminal domain I (from whole antiserum) (available from Sigma Aldrich® (P2318)) 89. A method according to any one of paragraphs 66 to 88 wherein the label bound to the antibody is a fluorochrome label.
90. A method according any one of paragraphs 66 to 89 wherein the label is selected from pacific blue (PacB), Horizon V450, pacific orange (PacO), AMCA, AmCyan, fluorescein isothiocyanate (FITC), Alexa488, phycoerythrin (PE), peridinin chlorophyl protein/cyanine 5.5 (PerCP-Cy5.5), PerCP, PE TexasRed, phycoerythrin/cyanine7 (PE-Cy7), allophycocyanine (APC), Alexa647, allophycocyanine/H7 (APC-H7), APC-Cy7, Alexa680 or Alexa700.
91. A method according to any one of paragraphs 66 to 90 wherein when more than one type of antibody is used, each type of antibody has a label which differs to the label of the other types of antibody present.
92. A method according to any one of paragraphs 66 to 91 wherein the bacterial toxin comprises pneumolysin and the antibody specific for pneumolysin is Ply-4 (IgG1 isotype).
93. A method according to any one of paragraphs 66 to 92 wherein the bacterial toxin comprises pneumolysin and the antibody specific for pneumolysin is Ply-4 (IgG1 isotype) conjugated to allophycocyanin.
94. A method according to any one of paragraphs 66 to 93 wherein the analysis of step (iii) is performed using flow cytometry.
95. A method according to any one of paragraphs 66 to 94 wherein the analysis of step (iii) is performed using multi-colour flow cytometry.
96. A method according to any one of paragraphs 66 to 95 wherein step (iii) further comprises comparing the results of the analysis to data generated using known concentrations of bacterial toxin in order to quantify the presence of bacterial toxin.
97. A liposome comprising cholesterol and a sphingmyelin wherein the liposomes have a diameter of between 1 and 2 μM.
98. A liposome according to paragraph 97 wherein the liposome is multilamellar.
99. A liposome according to any one of paragraphs 97 to 98 wherein the ratio of cholesterol to sphingomyelin is about 2:1.
100. A liposome according to any one of paragraphs 97 to 99 which comprises about 66 mol. % of cholesterol.
101. A conjugate comprising a liposome according to any one of paragraphs 97 to 100 and a bacterial toxin.
102. A conjugate according to paragraph 101 wherein the bacterial toxin is a bacterial toxin capable of binding to eukaryotic cell membranes.
103. A conjugate according to any one of paragraphs 101 to 102 wherein the bacterial toxin is a bacterial toxin capable of binding to mammalian cell membranes.
104. A conjugate according to any one of paragraphs 101 to 103 wherein the bacterial toxins is a bacterial toxin capable of binding to human cell membranes.
105. A conjugate according to any one of paragraphs 101 to 104 wherein the bacterial toxin is involved in the aetiology of one or more of sepsis, pneumonia, meningitis and urinary tract infections.
106. A conjugate according to any one of paragraphs 101 to 105 wherein the bacterial toxin is involved in the aetiology of sepsis.
107. A conjugate according to any one of paragraphs 101 to 106 wherein the bacterial toxin is derived from one or more of Streptococcus pneumoniae, Staphylococcus aureus, Escherichia coli, Klebsiella pneumoniae, Pseudomonas aeruginosa and Streptococcus pyogenes.
108. A conjugate according to any one of paragraphs 101 to 107 wherein the bacterial toxin is selected from pneumolysin (from Streptococcus pneumoniae), alpha haemolysin (from Staphylococcus aureus and/or Escherichia coli), haemolysin (from Klebsiella pneumoniae), exotoxin A (from Pseudomonas aeruginosa) and streptolysin O (from Streptococcus pyogenes).
109. A complex comprising a conjugate according to any one of paragraphs 101 to 108 and an antibody.
110. A complex according to paragraph 109 wherein the antibody is selected from at least one of Pneumolysin mouse monoclonal antibody Ply-4 (IgG1 isotype) (available from Abcam® (ab71810)), Lysteriolysin rabbit polyclonal antibody (IgG isotype) (available from Abcam® (ab200538)), Streptolysin mouse monoclonal antibody 6D11 (IgG1 isotype) (available from Abcam® (ab23501)), Alpha hemolysin mouse monoclonal antibody 8B7 (IgG1 isotype) (available from Abcam® (ab190467)), Tetanolysin mouse monoclonal antibody TetE3 (IgG1 isotype) (available from Abcam® (ab64755)), Exotoxin A rabbit polyclonal (from Pseudomonas aeruginosa) N terminal domain I (from whole antiserum) (available from Sigma Aldrich® (P2318)).
111. A complex according to any one of paragraphs 109 to 110 wherein the label bound to the antibody is a fluorochrome label.
112. A complex according to any one of paragraphs 109 to 111 wherein the label is selected from pacific blue (PacB), Horizon V450, pacific orange (PacO), AMCA, AmCyan, fluorescein isothiocyanate (FITC), Alexa488, phycoerythrin (PE), peridinin chlorophyl protein/cyanine 5.5 (PerCP-Cy5.5), PerCP, PE TexasRed, phycoerythrin/cyanine7 (PE-Cy7), allophycocyanine (APC), Alexa647, allophycocyanine/H7 (APC-H7), APC-Cy7, Alexa680 or Alexa700.
113. A kit for detecting the presence of bacterial toxins comprising:
(i) a container comprising liposomes and optionally a buffer, wherein the liposomes comprise a lipid capable of binding to one or more bacterial toxins; and
(ii) a container comprising a reagent, wherein the reagent comprises at least one type of antibody bound to a label; and
(iii) optionally further containers comprising a reagent, wherein the reagent comprises at least one type of antibody bound to a label;
wherein each type of antibody in the kit is specific for a different bacterial toxin to be detected.
114. A kit according to paragraph 113 wherein the liposomes comprise cholesterol and a sphingmyelin.
115. A kit according to paragraph 114 wherein the ratio of cholesterol to sphingomyelin is about 2:1.
116. A kit according to any one of paragraphs 113 to 115 wherein the liposome is multilamellar.
117. A kit according to any one of paragraphs 113 to 116 wherein the liposome comprises about 66 mol. % of cholesterol.
118. A kit according to any one of paragraphs 113 to 117 wherein the antibody is selected from at least one of Pneumolysin mouse monoclonal antibody Ply-4 (IgG1 isotype) (available from Abcam® (ab71810)), Lysteriolysin rabbit polyclonal antibody (IgG isotype) (available from Abcam® (ab200538)), Streptolysin mouse monoclonal antibody 6D11 (IgG1 isotype) (available from Abcam® (ab23501)), Alpha hemolysin mouse monoclonal antibody 8B7 (IgG1 isotype) (available from Abcam® (ab190467)), Tetanolysin mouse monoclonal antibody TetE3 (IgG1 isotype) (available from Abcam® (ab64755)), Exotoxin A rabbit polyclonal (from Pseudomonas aeruginosa) N terminal domain I (from whole antiserum) (available from Sigma Aldrich® (P2318)).
119. A kit according to any one of paragraphs 113 to 118 wherein the label bound to the antibody is a fluorochrome label.
120. A kit according to any one of paragraphs 113 to 119 wherein the label bound to the antibody is selected from pacific blue (PacB), Horizon V450, pacific orange (PacO), AMCA, AmCyan, fluorescein isothiocyanate (FITC), Alexa488, phycoerythrin (PE), peridinin chlorophyl protein/cyanine 5.5 (PerCP-Cy5.5), PerCP, PE TexasRed, phycoerythrin/cyanine7 (PE-Cy7), allophycocyanine (APC), Alexa647, allophycocyanine/H7 (APC-H7), APC-Cy7, Alexa680 or Alexa700.
121. A kit according to any one of paragraphs 113 to 120 wherein when more than one type of antibody is present in the kit, each type antibody has a label which differs to the label of the other types of antibody present in the kit.

### EXAMPLES

### Liposome Synthesis

In a class II microbiological cabinet 13.5 mg of cholesterol (Sigma Aldrich®) and 0.68 mg of sphingomyelin (Sigma Aldrich®) were mixed in 1 ml of chloroform (Sigma Aldrich®) (66% Cholesterol:Sphingomyelin). The solution underwent vacuum evaporation in an oxygen-free environment (nitrogen gas at 0.02 Bar) for 30 minutes. The dry lipid film formed was hydrated with 250 mM of 6-Carboxyfluorescein (Sigma Aldrich®). The mixture was incubated at 55° C. with constant vortexing for 1 hour to allow formation of giant multilamellar liposomes of between 1 and 2 μM diameter.

The liposome mixture was purified from the 6-Carboxyfluorescein by affinity chromatography (PD MiniTrap G-25 column prepacked with Sephadex® G-25 medium purchased from GE Healthcare® and Sigma Aldrich®). 200 μl of the liposome mixture were loaded on the column and 1.5 ml of PBS was used to allow the mixture to run through the column. For the elution of the mixture, an additional 1 ml of PBS was added in the column, achieving the desired concentration of 2 mg/ml.

Extrusion of multilamellar liposomal suspensions was achieved by using membranes with a pore size 1.0. μm. This pore size allows for a polydisperse suspension of multilamellar liposomes. The membranes and filter supports used are only for single use during liposome preparation.

The extruder stand/heating block was placed onto a hot plate and the temperature was monitored with a thermometer inside the heating block. The lipid mixture was hydrated, and underwent 3-5 freeze/thaw cycles by alternately placing the sample vial in a dry ice bath and warm water bath. The sample was then loaded into a gas-tight syringe and carefully place into one end of the mini-extruder. The temperature of the lipid suspension was allowed to equilibrate with the temperature of the heating block (approximately 5-10 minutes).

By pushing the plunger of the filled syringe the lipid solution will completely be transferred to the alternate syringe. Similarly the plunger of the alternate syringe was pushed to transfer the solution back to the original syringe. The lipid mixture should undergo 10 passes through the membrane. In general, the more passes though the membrane, the more homogenous the lipid solution becomes. The final extrusion will fill the alternate syringe in an effort to reduce the chances of contamination with larger particles or foreign material.

### Stability

The fluorescein liposomes are stable for 2 years under argon at 4° C. or for 3 months at 4° C. at atmospheric conditions.

### Pneumolysin Antibody Selection and Conjugation

The antibody used in this assay was a mouse monoclonal antibody to pneumolysin (PLY-4) (IgG1 isotype) and was purchased from Abcam® (ab71810). The PLY-4 antibody was conjugated with allophycocyanin (APC) using the Lightning-Link® allophycocyanin conjugation kit (purchased by Innova Biosciences).

### Preparation of Healthy Donor Plasma

Blood was collected into a citrate vacutainer and left to stand at room temperature for 1 hour. The vacutainers were then centrifuged at 2000×g for 20 mins. Plasma was then aliquoted and stored at −80° C.

### Patient Plasma Samples

All patient plasma samples came from the Royal Liverpool University Hospital and were obtained from patients with a clinical diagnosis of sepsis, a positive blood culture result with either Streptococcus pneumoniae or Haemophilus influenzae, and, in the case of S. pneumoniae, a semi-quantifiable infectious burden based upon results of a LytA qPCR. Blood was taken into a citrate vacutainer and plasma prepared as described above.

Pneumolysin Detection Using PLY-4/APC Liposomes with a Fluorescence-Activated Cell Sorting Assay

For the detection of pneumolysin with PLY-4/APC liposomes, 1 μg/ml of fluorescein-liposomes were incubated with 200 μl sepsis patient plasma sample or with various concentrations of purified pneumolysin in plasma purified from a healthy donor (pneumolysin concentration used: 2 μg, 1 μg, 0.5 μg, 0.2 μg, 0.1 μg, 0.05 μg, 0.01 μg and 0.005 μg). Incubation was carried out for 30 minutes at 4° C. Liposomes were then pelleted by centrifugation at 13,000×g for 10 minutes, were washed once in sterile PBS and then resuspended in 200 μl sterile PBS containing 1 μg/ml of PLY-APC. Incubation was carried out for 30 minutes at 4° C.

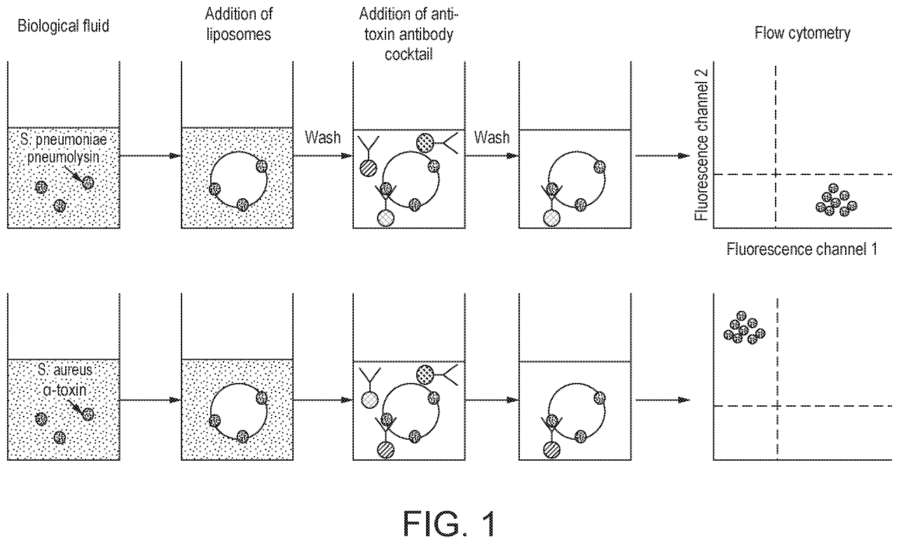

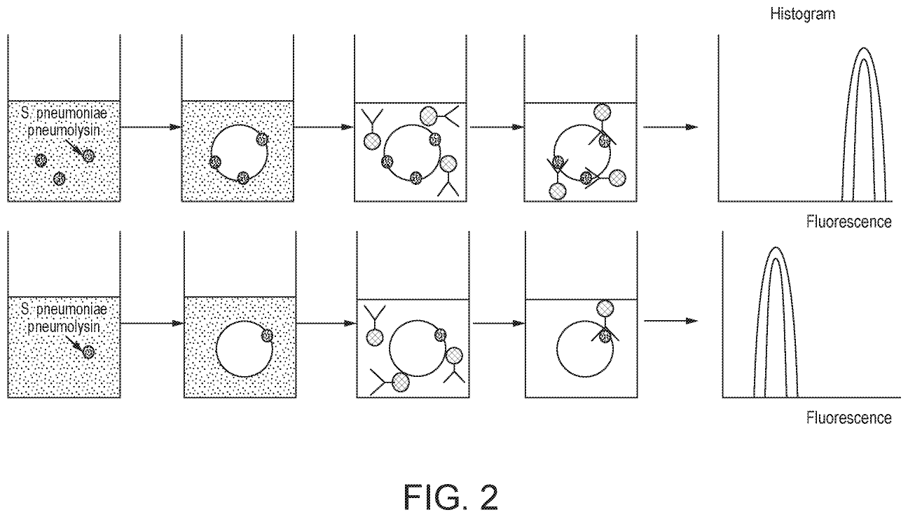

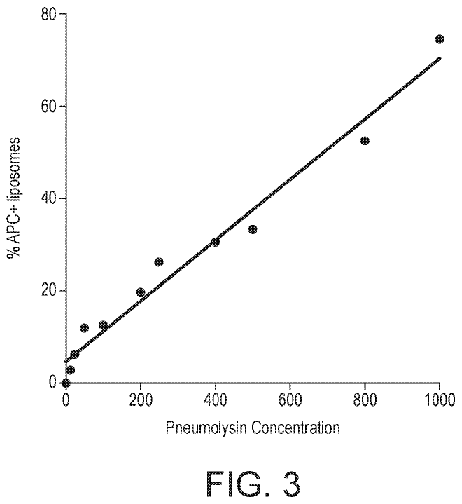

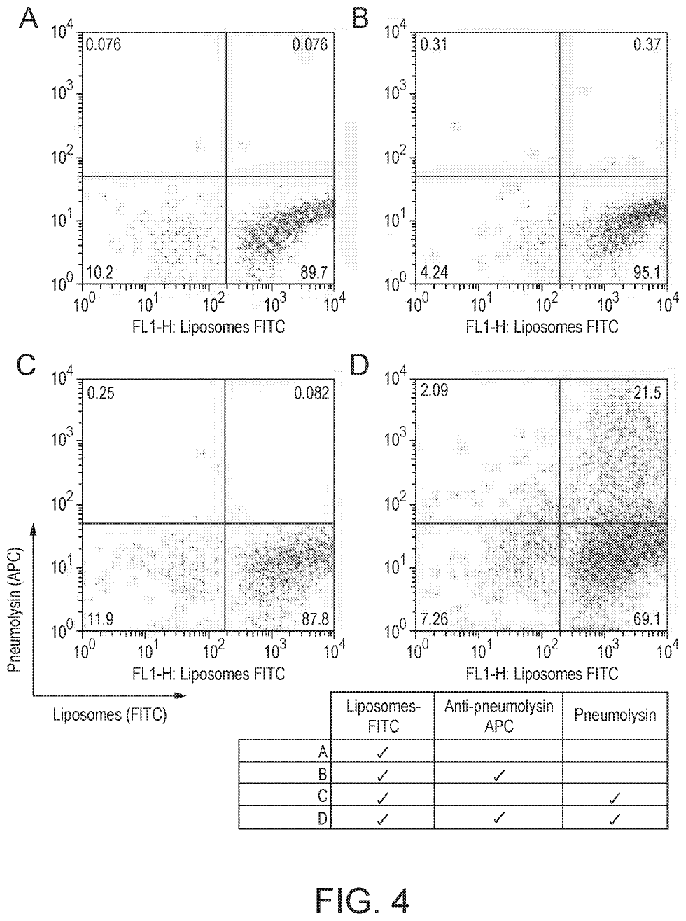

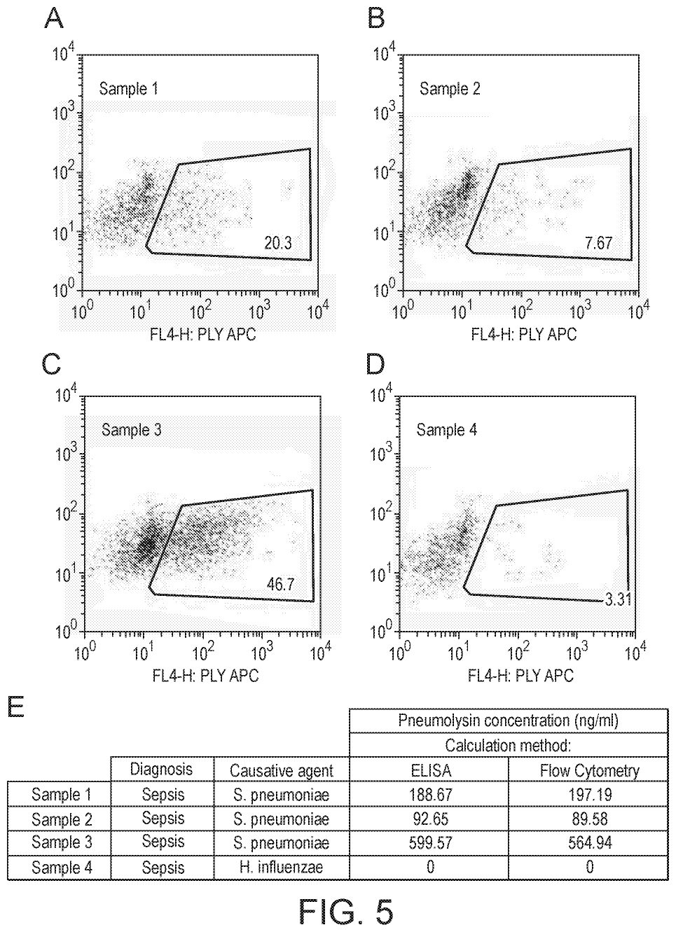

Post incubation of the toxin with the PLY-APC and the fluorescein-liposomes, the mixture was centrifuged at 14,000 rpm for 10 minutes, the supernatant of the tube was discarded and the pellet was re-suspended with 500 μl sterile PBS. After a brief vortexing, the samples were analysed on a BD FACSCalibur flow cytometer running Cell Quest acquisition software. The percentage of fluorescein (liposome) positive, APC (pneumolysin) positive events was recorded and unknown samples compared with a standard curve produced from the samples containing known pneumolysin concentrations. Samples containing liposomes and antibody but no pneumolysin, or liposomes and pneumolysin with no antibody were used as negative controls and for blank subtraction. An overview of this assay is shown in FIG. 1, standard curve generation is summarised in FIGS. 2 and 3. FIGS. 4 and 5 give example assay outputs. FIG. 4 shows that anti-pneumolysin antibody does not bind directly to liposomes in the absence of toxin (panel B), that pneumolysin does not lyse liposomes over the course of the assay (panel C) and that pneumolysin does not induce non-specific fluorescence (panel C). Panel D shows that the addition of purified pneumolysin to the assay induces a fluorescent shift in the APC channel as anti-pneumolysin APC antibodies bind to pneumolysin embedded in the liposome membrane. In this example, 21.5% of FITC+liposomes have been bound by pneumolysin.

If fluorescence compensation is required to correct for spectral overlap between different antibody-fluorochrome conjugates. This can be achieved as follows:
Use standard commercially available beads to ensure the cytometer is performing within specifications.
Set PMT voltages with an unstained/unlabelled liposome sample.
Adjust forward and side scatter so that liposome population is clearly observed.
Compensation controls are analysed for each fluorochrome in the assay. Positive and negative populations are used for each. Positive fluorescence is adjusted to be between 10\hat{ }3 and 10\hat{ }4 units. Compensation is correct when the median of the negative population is equal to the median of the positive population in the spill over channel.

### Comparison of Pneumolysin Detection by Fluorescein-Liposome Based Flow Cytometry and Enzyme-Linked Immunosorbent Assay.

A high-binding 96-well plate (Costar®) was coated with 1 μg/well PLY-4 antibody and incubated overnight, at 4° C. The plate was washed with 0.05% TWEEN-20 in PBS (purchased from Sigma Aldrich®) and then blocked for 2 hours with PBS 1% BSA (Sigma Aldrich®) at room temperature. Sepsis patient plasma samples or recombinant pneumolysin in healthy donor plasma was added to the wells. The pneumolysin was spiked into healthy donor plasma in a series of two-fold dilutions (100 to 1.56 ng/ml). The plate was incubated for 2 hours at room temperature. After 5× washes in PBS 0.05% TWEEN-20, 1 μg of PLY polyclonal antibody (Rabbit polyclonal to pneumolysin, IgG isotype purchased from Abcam® ab71811) was then added to each well in PBS and the plate was incubated at room temperature for 1 hour. Plate wells were washed, as above, and anti-rabbit IgG alkaline phosphatase (purchased from Abcam® ab6722) was added to each well and the plate was incubated for 30 minutes at room temperature. To allow the colour to develop, para-Nitrophenylphosphate (PNPP—purchased from Sigma Aldrich®) was added in the wells and the plate was incubated for 30 minutes in room temperature. Colour development was assessed by absorbance at 405 nm using a ThermoScientific Multiskan plate-reader running the Skanit for Multiskan Spectrum 2.2 software. Example ELISA-calculated pneumolysin concentrations in patient samples can be seen in FIG. 5E. The same samples were also tested by the liposome flow cytometry method (FIG. 5A-D) and gave comparable results (FIG. 5E).

Patient samples were analysed by ELISA methodology both before and after incubation with liposomes. A 100 μl aliquot of patient sample was incubated with 1 μg/ml fluorescein-liposomes for 30 minutes at 4° C. Liposomes were then pelleted by centrifugation as described and the plasma supernatant used in the pneumolysin-detection ELISA and results compared to the matched patient sample which had not been treated with liposomes. The pelleted liposomes were used in the flow cytometry assay to detect bound pneumolysin.

### Pneumolysin Detection Over the Course of Pneumococcal Sepsis in Mice.

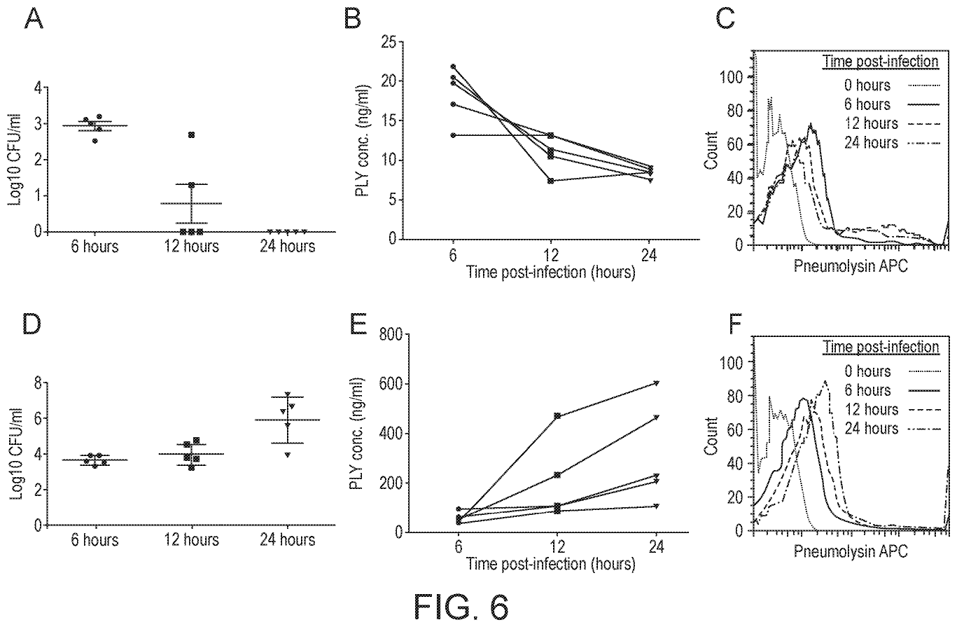

Mice were infected intravenously via the tail vein with 1×10⁶ colony forming units (CFU) of S. pneumoniae serotype 23F or serotype 2 strain D39 in 100 ul phosphate buffered saline (PBS). Blood samples were taken at 0, 6, 12 and 24 hours post-infection by withdrawal of 10 μl blood from a superficial vessel and the infectious burden determined by serial dilution of blood onto blood agar and enumeration of colonies after overnight incubation at 37° C. See FIG. 6A (serotype 23F) and FIG. 6D (serotype 2 strain D39).

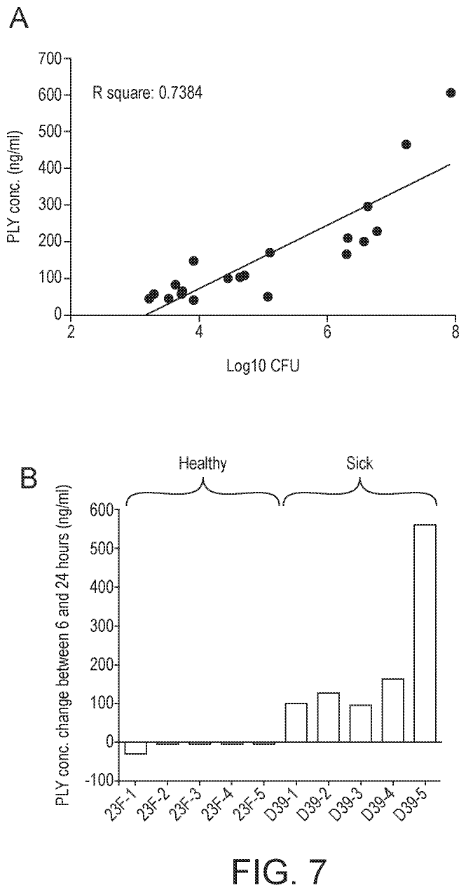

Pneumolysin-concentrations in mouse serum were determined by flow cytometry following incubation with fluorescein-liposomes and addition of APC-conjugated anti-pneumolysin antibody. See FIGS. 6B and 6C (serotype 23F) and FIGS. 6E and 6F (serotype 2 strain D39). Assay calculated toxin concentrations correlated with infectious burden and disease outcome. Self-resolving infection with 23F saw declining bacterial numbers (FIG. 6A) and toxin concentrations (FIGS. 6B and C) over time, whilst lethal D39 infection saw increasing infection burden (FIG. 6D) and increasing toxin concentration (FIGS. 6E and F). Data from 5 mice at four different time points showed that assay calculated blood toxin concentrations correlated with the density of bloodstream infection. See FIG. 7A. Furthermore, changes in blood pneumolysin concentration over time were found to correlate with disease outcome. The five mice infected with serotype 23F say decreasing pneumolysin concentrations in blood between 6 and 24 hours and went on to clear infection. The mice infected with serotype 2 strain D39 had increasing pneumolysin concentrations and were culled due to ill health.

### Group a Streptococcus Detection in Mice

Mice were infected intravenously via the tail vein with 1×10⁷ colony forming units (CFU) of S. pyogenes Merseyside outbreak strain 112327 (emm type 32.2) IN 50 μl PBS. Blood samples (10 ul) were withdrawn from superficial vessels at 0, 6, 12 and 24 hours post-infection and the infectious burden determined by serial dilution of blood onto blood agar and enumeration after overnight incubation at 37° C.

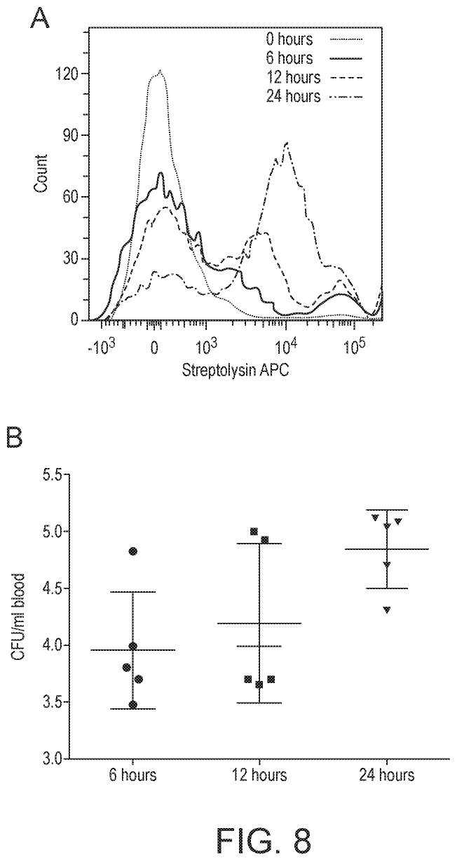

FIG. 8A shows the mean fluorescence intensity of liposomes and anti-streptolysin APC antibody incubated with serum taken from Streptococcus pyogenes infected mice at 0 (green), 6 (orange), 12 (blue) or 24 (red) hours post-infection. FIG. 8B reports Streptococcus pyogenes colony forming units (CFU) per ml of blood. MFI toxin concentration) increases over time (FIG. 8A), as does CFU (FIG. 8B).

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise paragraphed. No language in the specification should be construed as indicating any non-paragraphed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the paragraphs appended hereto as permitted by applicable law.

1. A method for detecting the presence in a biological fluid of one or more bacterial toxins capable of binding to cell membranes, wherein the method comprises
(i) incubating the biological fluid with a plurality of liposomes, wherein the liposomes comprise a lipid capable of binding to said one or more toxins, to provide one or more liposome-toxin conjugates;
(ii) incubating said one or more conjugates with at least one type of antibody bound to a label to provide one or more conjugate-antibody complexes;
wherein, each type of said at least one type of antibody is specific for one of the one or more bacterial toxins whose presence is to be detected; and
(iii) analysing said one or more complexes in order to detect the presence of one or more bacterial toxins capable of binding to cell membranes.

2. The method of claim 1, wherein the method is an in vitro method.

3. The method of claim 1, wherein the biological fluid is human biological fluid.

4. The method of claim 1, wherein the biological fluid is selected from one of whole blood, blood plasma, blood serum, CSF, and urine.

5. The method of claim 1, wherein the one or more bacterial toxins are involved in the aetiology of one or more of sepsis, pneumonia, meningitis, and urinary tract infections.

6. The method of claim 1, wherein the one or more bacterial toxins are selected from pneumolysin (from Streptococcus pneumoniae), alpha haemolysin (from Staphylococcus aureus and/or Escherichia coli), haemolysin (from Klebsiella pneumoniae), exotoxin A (from Pseudomonas aeruginosa), and streptolysin O (from Streptococcus pyogenes).

7. The method of claim 1, wherein the liposomes comprise cholesterol and sphingomyelin.

8. The method of claim 1, wherein the liposomes comprise about 50 mol. % to about 66 mol. % of cholesterol.

9. The method of claim 1, wherein the liposomes have a diameter of about 1.0 μm to about 2 μm.

10. The method of claim 1, wherein the at least one type of antibody is selected from at least one of Pneumolysin mouse monoclonal antibody Ply-4 (IgG1 isotype) (available from ABCAM® (ab71810)), Lysteriolysin rabbit polyclonal antibody (IgG isotype) (available from ABCAM® (ab200538)), Streptolysin mouse monoclonal antibody 6D11 (IgG1 isotype) (available from ABCAM® (ab23501)), Alpha hemolysin mouse monoclonal antibody 8B7 (IgG1 isotype) (available from ABCAM® (ab190467)), Tetanolysin mouse monoclonal antibody TetE3 (IgG1 isotype) (available from ABCAM® (ab64755)), and Exotoxin A rabbit polyclonal (from Pseudomonas aeruginosa) N terminal domain I (from whole antiserum) (available from SIGMA ALDRICH® (P2318)).

11. The method of claim 1, wherein the label bound to the antibody is a fluorochrome label.

12. The method of claim 1, wherein the label is selected from pacific blue (PacB), Horizon V450, pacific orange (PacO), AMCA, AmCyan, fluorescein isothiocyanate (FITC), Alexa488, phycoerythrin (PE), peridinin chlorophyl protein/cyanine 5.5 (PerCP-Cy5.5), PerCP, PE TexasRed, phycoerythrin/cyanine7 (PE-Cy7), allophycocyanine (APC), Alexa647, allophycocyanine/H7 (APC-H7), APC-Cy7, Alexa680, and Alexa700.

13. The method of claim 1, wherein when more than one type of antibody is used, each type of antibody has a label which differs from the label of the other types of antibody present.

14. The method of claim 1, wherein the bacterial toxin comprises pneumolysin and the antibody specific for pneumolysin is Ply-4 (IgG1 isotype) conjugated to allophycocyanin.

15. The method of claim 1, wherein the analysis of step (iii) is performed using multi-colour flow cytometry.

16. A complex comprising:
(i) a conjugate comprising a liposome and a bacterial toxin; and
(ii) an antibody bound to a label.

17. The complex of claim 16, wherein the label bound to the antibody is a fluorochrome label.

18. A kit for detecting the presence of bacterial toxins comprising:
(i) a first container comprising liposomes, wherein the liposomes comprise a lipid capable of binding to one or more bacterial toxins; and
(ii) a second container comprising a reagent, wherein the reagent comprises at least one antibody bound to a label;
wherein each type of antibody in the kit is specific for a different bacterial toxin to be detected.

19. The kit of claim 18, further comprising a third container comprising a reagent, wherein the reagent comprises at least one antibody bound to a label.
